(19) United States
(12) Patent Application Publication
(10) Pub. No.: US 2006/0057622 A1
(43) Pub. Date: Mar. 16, 2006

(54) Low cycle amplification of RNA molecules

(75) Inventors: Dennis A. Steindler, Gainesville, FL (US); Oleg N. Suslov, Gainesville, FL (US)

Correspondence Address: AKERMAN SENTERFITT, P.O. BOX 3188, WEST PALM BEACH, FL, 33402-3188, US

(73) Assignee: University of Florida, Gainesville, FL (US)

(21) Appl. No.: 11/212,996

(22) Filed: Aug. 25, 2005

Related U.S. Application Data
(60) Provisional application No. 60/604,262, filed on Aug. 25, 2004.

(51) Int. Cl. C12Q 1/68; C12P 19/34

(52) U.S. Cl. 435/6; 435/91.2

(57) ABSTRACT

The compositions and methods afford an almost unlimited linear RNA amplification from a few cells with minimal differences in the relative abundance of amplified RNAs and their parent mRNA (sample distortion).

This application claims the benefit of provisional patent application U.S. Ser. No. 60/604,262 filed Aug. 25, 2004 which is incorporated by reference, herein, in its entirety.

### FIELD OF THE INVENTION

The invention relates to methods for determining molecular mechanisms underlying stem cells self-renewal and differentiation into specific cell lineages that can generate different tissues. In particular, methods for determining low copy gene expression profiling of stem cells, are provided.

### BACKGROUND OF THE INVENTION

Despite extensive phenotypic and functional characterization of stem and progenitor cells, little is known about the molecular nature of regulatory mechanisms governing their proliferation, fate choice, and differentiation. Gene expression profiling of human stem/progenitor cell clones has revealed a clear cut heterogeneity of clone-forming cells. This heterogeneity demands a need for gene expression profiling of individual cells that affords markers not only for undifferentiated cells but also for central nervous system (CNS) stem cells and committed but undifferentiated progenitors.

A prerequisite for such profiling is a need for an RNA amplification method from a finite cell population or even from an individual cell. The two most widely used methods with a myriad of modifications are SMART (switch mechanism at the 5′ ends of RNA templates), the PCR technique, and antisense RNA amplification method that was developed by Dr. J. Eberwine and collaborators. The main disadvantage of the PCR-based technique is that cDNAs of different length and composition are amplified with different efficiencies. Although similar types of problems should be considered with the antisense technology, any differences with this amplification technique will be linear rather than exponential (as occurs with PCR). There is an inherent bias to preferentially amplify the 3′ end of mRNAs during the aRNA procedure. This complicates the characterization of new genes and the detection of alternatively spliced isoforms since the 3′ UTR region may provide little or no information on their identity until a longer or full-length clone can be isolated.

mRNA profiling has become, within a relatively short period of time, an invaluable tool for the simultaneous analysis of mRNA expression levels for up to thousands of genes upon physiological, developmental, and pathological processes, as well as in cellular/genetic responses to drug treatment. Nowhere is the appropriate use of molecular markers more important than in the study of a stem cell molecular signature, and during cell fate-specification and cellular differentiation. There is a need to distinguish the transition from one cellular phenotype to another. When these molecular data are combined with electrophysiological, morphological, immunohistochemical, and anatomical analyses, a detail portrait of cell genesis, fate choice, and precise phenotypic differentiation can be obtained. Thus, there is an urgent need in the art for identification of genes, especially those genes in low copy numbers.

### SUMMARY OF THE INVENTION

The invention provides methods and compositions for amplification of nucleic acid molecules. In particular, the invention provides for amplification of RNA with few errors and alleviates problems with RNA degradation. The methods disclosed herein, can provide crucial insights into stem cell regulation via uncovering interacting signaling pathways. The invention may be used against protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement of, for instance immunoglobulin genes, etc.

In a preferred embodiment, the invention provides methods for amplifying isolated nucleic acids from a sample, such as a stem cell. Preferably, any isolated nucleic acid molecule is amplified including, but not limited to low copy number or rare genes. Parameters for determination of copy numbers of genes include, but are not limited to the different length, structure and the abundance of specific transcripts.

In one preferred embodiment, candidate genes are identified to represent all groups of abundance: 1) abundant (about 1,000-3,000 copies per cell); 2) moderately expressed genes (about 300-1,000 copies per cell); 3) rare genes (less than about 300 copies per cell). Each group should comprise the transcripts which differ in the length (small, medium, long). Examples of candidate genes in different groups include, for example, group of abundant genes: actin (the length is 1,800 nt; 3,200 copies per cell) and tenascin (the length is 7,500 nt; 1,100 copies per cell); rare gene candidates: TBP (the length is 1,900 nt; 76 copies per cell) and TFRC (the length is 5,000 nt, 160 copies per cell). Preferably, copy number is calculated based on the calibration curve of each given transcript. For example, full-length transcripts are amplified by PCR using proof-reading DNA Polymerase which allows monitoring 5′/3′ ratio of amplified products.

In another preferred embodiment, amplification of nucleic acid molecules from a sample comprises the steps of: (a) isolating nucleic acid molecules from a sample; (b) providing a modified oligo-dT primer and SWITCH primer comprising an RNA polymerase site; (c) hybridizing said isolated nucleic acid sample and said primers; (d) administering polymerase for amplification in a first-strand synthesis reaction, wherein, said polymerase adds nucleotides to 3′ ends of transcribed cDNA providing complementary nucleotides for the SWITCH primer; and, (e) creating an extended template for said SWITCH primer wherein said polymerase switches templates and amplifies said template; thereby, (f) providing full-length, single stranded cDNA comprising a complete 5′ end of isolated nucleic acid, as well as sequences that are complimentary to the T7-SWITCH oligonucleotide; and, cycling of steps (e) through (f) thereby, amplifying said genes. Preferably, a sequence specific primer and/or universal base primer are administered to the amplification method at step (b).

In one preferred embodiment, the isolated nucleic acid is RNA and the polymerase is a RNA polymerase, such as for example, T7 RNA polymerase. Preferred primers include, but are not limited to a target nucleic acid specific primer and/or a universal primer. Preferably, a SWITCH primer is used such as, for example a T7-SWITCH primer comprising a T7 RNA polymerase site and/or anchor site.

In another preferred embodiment, nucleotides are inserted at the 3′ end of the cDNA. In accordance with the invention, when reverse transcriptase reaches the 5′ end of mRNA, the enzyme's terminal transferase activity inserts (adds) a few additional nucleotides, primarily deoxycitidine, to the 3′ end of the cDNA. Preferably, the polymerase inserts at least one additional nucleotide at 3′ ends of transcribed mRNA, more preferably, the polymerase inserts about two additional nucleotides at 3′ ends of transcribed mRNA; more preferably, the polymerase inserts about 5 additional nucleotides at 3′ ends of transcribed mRNA, more preferably, the polymerase inserts about 10 additional nucleotides at 3′ ends of transcribed mRNA, more preferably, the polymerase inserts from about 5 up to 20 additional nucleotides at 3′ ends of transcribed mRNA.

In accordance with the invention, the inserted nucleotides at the 3′ ends of transcribed mRNA are deoxycitidine, however, any nucleobase is within the scope of the invention. Preferably, the SWITCH primer comprises an oligonucleotide sequence at its 3′ end and the oligonucleotide sequence is complementary to the inserted nucleotides at the 3′ ends of transcribed cDNA. For example, if the reverse transcriptase inserts deoxycitidine, the SWITCH primer comprises a guanisine (rG) at the 3′ end, allowing for base-pairing between the transcribed nucleic acid product and SWITCH primer. Preferably, the 3′ end of the SWITCH primer comprises about 1, 2, 3, 4, 5, 6, 7, 8, 9, 10 up to 15 guanosines (rG). In a preferred embodiment, the SWITCH primer comprises three guanosines (rG). In another preferred embodiment, the 3′ end of the SWITCH primer comprises about 1, 2, 3, 4, 5, 6, 7, 8, 9, 10 modified guanosines.

In another preferred embodiment, the modified oligo-dT primer comprises about 1, 2, 3, 4, 5, 6, 7, 8, 9, 10, or comprises only modified base units. Preferably, the modified oligo-dT primer comprises any one or combinations thereof, of phosphorthiorate, methylphosphonate, peptide nucleic acids, and LNA molecules. The modified oligo-dT primer can comprise between about two bases up to twenty bases.

Preferably, the modified oligo-dT primer comprises at least one modified nucleobase, more preferably, the modified oligo-dT primer comprises about two modified nucleobases, more preferably, the modified oligo-dT primer comprises about five modified nucleobases, more preferably, the modified oligo-dT primer comprises about ten modified nucleobases.

In another preferred embodiment, the modified oligo-dT primer comprises about 1, 2, 3, 4, 5, 6, 7, 8, 9, 10, or comprises only modified base units. Preferably, the modified oligo-dT primer comprises any one or combinations thereof, of phosphorthiorate, methylphosphonate, peptide nucleic acids, and LNA molecules.

In another preferred embodiment, the modified oligo-dT primer comprises between about two bases up to fifty nucleotide bases.

In another preferred embodiment, the SWITCH primer comprises a 3′ oligonucleotide stretch of guanosines (rG) of at least one nucleotide base up to twenty nucleotide bases. More preferably, the SWITCH primer comprises a 3′ oligonucleotide stretch of guanosines (rG) three (rG) nucleotide bases.

In another preferred embodiment, the 3′ end of the SWITCH primer comprises about 1, 2, 3, 4, 5, 6, 7, 8, 9, 10 up to 15 guanosines (rG). In accordance with the invention, the 3′ end of the SWITCH primer can comprise about 1, 2, 3, 4, 5, 6, 7, 8, 9, 10 modified guanosines (rG). Preferably, the SWITCH primer comprises at least one nucleotide bases up to fifty nucleotide bases. In accordance with the invention the SWITCH primer comprises a total of about 38 nucleotide bases.

In another preferred embodiment, the T7-SWITCH anchor sequence and poly A sequence are universal priming sites for end-to-end cDNA amplification via long-distance PCR. Preferably, the PCR method disclosed herein, comprises PCR cycles of isolated nucleic acid sequences are between about 10 to 20 cycles. Preferably, one cycle of amplification yields between about 1.7×10⁴-8.3×10⁴ fold of amplified product as compared to controls which have not been subjected to amplification, and, two cycles of amplification yields between about 5.8×10⁶-2.4×10⁷ fold of amplified product as compared to controls which have not been subjected to amplification. Thus, according to the invention there is an exponential increase in yields of amplified product and which is not subjected to the number of cycles used by one of ordinary skill in the art.

In another preferred embodiment, the yields of the nucleic acids are quantified by any standard method known to one of ordinary skill in the art, such as for example, by U.V. readings.

In another preferred embodiment, the invention provides a composition for amplifying RNA molecules comprising: an isolated nucleic acid molecule from a sample; a modified oligo-dT primer and SWITCH primer comprising an RNA polymerase site; polymerase for amplification in a polymerase chain reaction.

In another preferred embodiment, a composition comprises: a modified oligo-dT primer and SWITCH primer comprising an RNA polymerase site; a polymerase for amplification in a first-strand synthesis reaction, wherein, said polymerase adds nucleotides to 3′ ends of transcribed cDNA providing complementary nucleotides for the SWITCH primer.

Preferably, the modified oligo-dT primer comprises at least one modified nucleobase, more preferably, the modified oligo-dT primer comprises about two modified nucleobases, more preferably, the modified oligo-dT primer comprises about five modified nucleobases, more preferably, the modified oligo-dT primer comprises about ten modified nucleobases.

In another preferred embodiment, the modified oligo-dT primer comprises about 1, 2, 3, 4, 5, 6, 7, 8, 9, 10, or comprises only modified base units. Preferably, the modified oligo-dT primer comprises any one or combinations thereof, of phosphorthiorate, methylphosphonate, peptide nucleic acids, and LNA molecules.

In another preferred embodiment, the modified oligo-dT primer comprises between about two bases up to fifty nucleotide bases.

In another preferred embodiment, the SWITCH primer comprises a 3′ oligonucleotide stretch of guanosines (rG) of at least one nucleotide base up to twenty nucleotide bases.

In another preferred embodiment, the 3′ end of the SWITCH primer comprises about 1, 2, 3, 4, 5, 6, 7, 8, 9, 10 up to 15 guanosines (rG). In accordance with the invention, the 3′ end of the SWITCH primer can comprise about 1, 2, 3, 4, 5, 6, 7, 8, 9, 10 modified guanosines (rG). Preferably, the SWITCH primer comprises at least five nucleotide bases up to fifty nucleotide bases. In accordance with the invention the SWITCH primer comprises a total of about 38 nucleotide bases.

In another preferred embodiment, the invention provides a kit, said kit comprising an isolated nucleic acid molecule from a sample; a modified oligo-dT primer and SWITCH primer comprising an RNA polymerase site; polymerase for amplification in a polymerase chain reaction. Preferably, instructions for use are included. Said instructions include a method, for example: amplification of nucleic acid molecules from a sample comprises the steps of: isolating nucleic acid molecules from a sample; providing a modified oligo-dT primer and SWITCH primer comprising an RNA polymerase site; hybridizing said isolated nucleic acid sample and said primers; administering polymerase for amplification in a first-strand synthesis reaction, wherein, said polymerase adds nucleotides to 3′ ends of transcribed cDNA providing complementary nucleotides for the SWITCH primer; and, creating an extended template for said SWITCH primer wherein said polymerase switches templates and amplifies said template; thereby, providing full-length, single stranded cDNA comprising a complete 5′ end of isolated nucleic acid, as well as sequences that are complimentary to the T7-SWITCH oligonucleotide; and, cycling of steps thereby, amplifying said genes. Preferably, a sequence specific primer and/or universal base primer are administered to the amplification of the sample nucleic acid. Universal primers are commercially available or can be synthesized.

Preferably, the modified oligo-dT primer comprises at least one modified nucleobase, more preferably, the modified oligo-dT primer comprises about two modified nucleobases, more preferably, the modified oligo-dT primer comprises about five modified nucleobases, more preferably, the modified oligo-dT primer comprises about ten modified nucleobases.

In another preferred embodiment, the modified oligo-dT primer comprises about 1, 2, 3, 4, 5, 6, 7, 8, 9, 10, or comprises only modified base units. Preferably, the modified oligo-dT primer comprises any one or combinations thereof, of phosphorthiorate, methylphosphonate, peptide nucleic acids, and LNA molecules.

In another preferred embodiment, the modified oligo-dT primer comprises between about two bases up to fifty nucleotide bases.

In another preferred embodiment, the SWITCH primer comprises a 3′ oligonucleotide stretch of guanosines (rG) of at least one nucleotide base up to twenty nucleotide bases.

In another preferred embodiment, the 3′ end of the SWITCH primer comprises about 1, 2, 3, 4, 5, 6, 7, 8, 9, 10 up to 15 guanosines (rG). In accordance with the invention, the 3′ end of the SWITCH primer can comprise about 1, 2, 3, 4, 5, 6, 7, 8, 9, 10 modified guanosines (rG). Preferably, the SWITCH primer comprises at least five nucleotide bases up to fifty nucleotide bases. In accordance with the invention the SWITCH primer comprises a total of about 38 nucleotide bases.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is an schematic diagram illustrating an example of the amplification method.

FIG. 2 is a gel showing the quality of isolated total RNA.

FIG. 3 is a graph showing the quality of isolated total RNA.

FIG. 4 is a graph showing the quality of isolated total RNA.

FIG. 5 is a graph illustrating the expression profile of a sample without amplification.

FIG. 6 is a graph illustrating the expression profile of 0.5 ng of total RNA from LN 229 after amplification.

FIG. 7 is a graph illustrating the expression profile of 1 ng of total RNA from LN 229 after amplification.

FIG. 8 is a graph illustrating the expression profile of 2 ng of total RNA from LN 229 after amplification.

FIG. 9 is a graph illustrating that one and two rounds of amplification yields similar amplification results based on the similarity of amplification approaches and the template's identity (sense RNA) used in the first and the second rounds.

FIG. 10 is a graph illustrating the expression profile of 20 ng of total RNA from LN 229 after amplification.

FIG. 11 is a graph illustrating that one and two rounds of amplification yields similar amplification results based on the similarity of amplification approaches and the template's identity (sense RNA) used in the first and the second rounds. The graph illustrates a second round of amplification of 5 ng total RNA from LN 229.

FIG. 12 is a graph illustrating that one and two rounds of amplification yields similar amplification results based on the similarity of amplification approaches and the template's identity (sense RNA) used in the first and the second rounds. (Small glioma sphere—1^{st} round of amplification). The graph also illustrates the distortion of amplification products from single glioma spheres from 21 cycles of PCR in comparison with 12 cycles.

FIG. 13 is a graph illustrating that one and two rounds of amplification yields similar amplification results based on the similarity of amplification approaches and the template's identity (sense RNA) used in the first and the second rounds. (Large glioma sphere—1^{st} round of amplification). The graph also illustrates the distortion of amplification products from single glioma spheres from 21 cycles of PCR in comparison with 12 cycles.

FIG. 14 is a graph illustrating that one and two rounds of amplification yields similar amplification results based on the similarity of amplification approaches and the template's identity (sense RNA) used in the first and the second rounds. (Small glioma sphere—2^{nd} round of amplification).

FIG. 15 is a graph illustrating that one and two rounds of amplification yields similar amplification results based on the similarity of amplification approaches and the template's identity (sense RNA) used in the first and the second rounds. (Large glioma sphere—2^{nd} round of amplification).

FIG. 16 is a graph illustrating the distortion of amplification products from single glioma spheres from 21 cycles of PCR in comparison with 12 cycles. (Small glioma sphere—1^{st} round of amplification).

FIG. 17 is a graph illustrating the distortion of amplification products from single glioma spheres from 21 cycles of PCR in comparison with 12 cycles. (Large glioma sphere—2nd round of amplification).

FIG. 18 shows an amplified population of transcripts using the disclosed method (lanes 2 and 3) which are longer in size and 5′ end-enriched, as compared to (lane 4) the modified Eberwine method (Ambion kit)

### DETAILED DESCRIPTION OF THE INVENTION

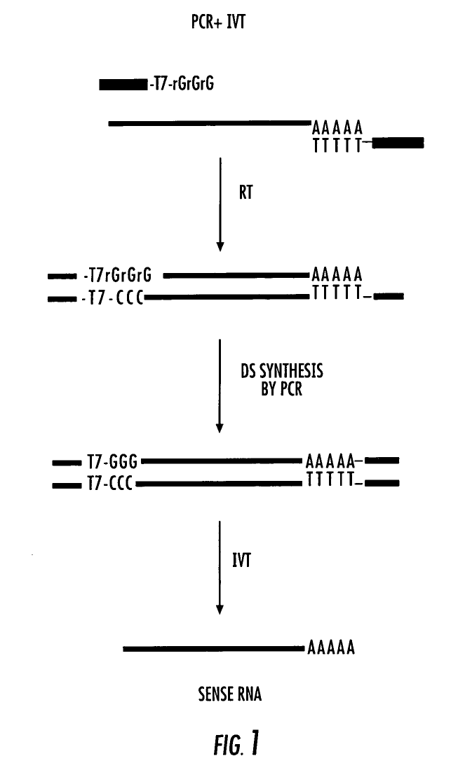

The RNA amplification methods disclosed herein, create a regenerating biorepository that represents the complex mRNA profile of the original sample (FIG. 1—flow chart). In particular, the methods exploit the template switching activity of reverse transcriptase (RT) to incorporate RNA polymerase binding sites upstream of single stranded DNA (ssDNA). Limited amounts of PCR cycles, as low as 12-13 cycles are used for the synthesis of double-stranded (dsDNA) in order to introduce minimal sample distortion.

There is the limited amount of total RNA presented in a single cell (20-30 pg). Current methods do not provide unlimited amplification since PCR has a natural limitation (the so-called “plateau effect”), and the aRNA method results in a 10⁵-10⁶-fold amplification of 3′ biased RNA after two rounds. Micrograms of RNA are needed for cDNA array screening, subtraction procedures, quantitative PCR, reverse Northerns, etc. The present method affords an almost unlimited linear RNA amplification from a few cells with minimal differences in the relative abundance of amplified RNAs and their parent mRNA (sample distortion).

Definitions

Prior to setting forth the invention the following definitions are provided:

As used herein, the terms “exon” and “intron” are art-understood terms referring to various portions of genomic gene sequences. “Exons” are those portions of a genomic gene sequence that encode protein. “Introns” are sequences of nucleotides found between exons in genomic gene sequences.

As used herein the terms “rare” or “low copy numbers” refer to nucleic acid molecules that are less than about 300 copies per cell. The terms “moderate” or “medium copy numbers” refer to nucleic acid molecules that are about 300-1,000 copies per cell. The terms “abundant” or “high copy numbers” refer to nucleic acid molecules that are about 1,000-3,000 copies per cell.

“Amplification” relates to the production of additional copies of a nucleic acid sequence.

As used herein, the singular forms “a”, “an” and “the” include plural referents unless the context clearly dictates otherwise.

A “universal” priming site is a site to which a universal primer will hybridize. In general, “universal” refers to the use of a single primer or set of primers for a plurality of amplification reactions. It should also be noted that “sets” of universal priming sequences/primers may be used. For example, in highly multiplexed reactions, it may be useful to use several sets of universal sequences, rather than a single set.

The terms “nucleic acid molecule” or “polynucleotide” will be used interchangeably throughout the specification, unless otherwise specified. As used herein, “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

As used herein, the term “oligonucleotide specific for” refers to an oligonucleotide having a sequence (i) capable of forming a stable complex with a portion of the targeted gene, e.g. by either strand invasion or triplex formation, or (ii) capable of forming a stable duplex with a portion of a mRNA transcript of the targeted gene a mechanism also called antisense.

As used herein, the terms “oligonucleotide” or “primers” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorthiorate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoögsteen types of base pairing, or the like.

The oligonucleotide may be composed of a single region or may be composed of several regions. For example, hinge regions comprising different lengths and base composition. The oligonucleotide may be “chimeric” or “modified”, that is, composed of different regions. In the context of this invention “chimeric” or “modified” compounds are oligonucleotides, which comprise two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties.

The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N₆-methyladenine, 7-deazaxanthine, 7-deazaguanine, N₄,N₄-ethanocytosin, N₆,N₆-ethano-2,6-diaminopurine, 5-methylcytosine, 5-(C₃-C₆)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139(1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000), ); 2′-O, 3′-C-linked [3.2.0] bicycloarabinonucleosides (see e.g. N. K Christiensen., et al., J. Am. Chem. Soc., 120: 5458-5463 (1998). Such analogs include synthetic nucleosides designed to enhance binding properties, e.g., duplex or triplex stability, specificity, or the like.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “Thermal Melting Point (Tm)” refers to the temperature, under defined ionic strength, pH, and nucleic acid concentration, at which 50% of the probes complementary to the target sequence hybridize to the target sequence at equilibrium. As the target sequences are generally present in excess, at Tm, 50% of the probes are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

As used herein, the terms “probe” or “capture probe” are defined as a nucleic acid, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids (PNA) in which the constituent bases are joined by peptide bonds rather than phosphodiester linkages.

The term “target nucleic acid” refers to a nucleic acid (often derived from a biological sample), to which the probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid may refer to the specific subsequence of a larger nucleic acid to which the probe is directed or to the overall sequence (e.g., gene or mRNA) whose expression level it is desired to detect. The difference in usage will be apparent from context.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target genes. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

As used herein, the term “mRNA” means the presently known mRNA transcript(s) of a targeted gene, and any further transcripts which may be elucidated.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Methods of alignment of sequences for comparison are well known in the art. Optimal alignment of sequences for comparison may be conducted by the local homology algorithm of Smith and Waterman, Adv. Appl. Math. 2: 482 (1981), by the homology alignment algorithm of Needleman and Wunsch J. Mol. Biol. 48: 443 (1970), by the search for similarity method of Pearson and Lipman, Proc. Natl. Acad. Sci. USA 85: 2444 (1988), by computerized implementations of these algorithms (including, but not limited to CLUSTAL in the PC/Gene program by Intelligenetics, Moutain View, Calif., GAP, BESTFIT, FASTA, and TFASTA in the Wisconsin Genetics Software Package, Genetics Computer Group (GCG), 575 Science Dr., Madison, Wis., USA), or by inspection. In particular, methods for aligning sequences using the CLUSTAL program are well described by Higgins and Sharp in Gene, 73: 237-244 (1988) and in CABIOS 5: 151-153 (1989)).

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., WO 92/08796, published May 29, 1992, and WO 94/28143, published Dec. 8, 1994).

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a substrate can comprise a multiplex adsorbent characterized by many different adsorbent species (e.g., anion exchange materials, metal chelators, or antibodies), having different binding characteristics. Substrate material itself can also contribute to adsorbing a nucleic acid molecule and may be considered part of an “adsorbent.”

“Adsorption” or “retention” refers to the detectable binding between an absorbent and a nucleic acid molecule either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the substrate surface.

“Detect” refers to identifying the presence, absence or amount of the object to be detected.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample.

“Energy absorbing molecule” or “EAM” refers to a molecule that absorbs energy from an ionization source in a mass spectrometer thereby aiding desorption of analyte from a surface. Depending on the size and nature of the analyte, the energy absorbing molecule can be optionally used. Energy absorbing molecules used in MALDI are frequently referred to as “matrix.” Cinnamic acid derivatives, sinapinic acid (“SPA”), cyano hydroxy cinnamic acid (“CHCA”) and dihydroxybenzoic acid are frequently used as energy absorbing molecules in laser desorption of bioorganic molecules.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

Any cell can be used in the methods of the invention, including but not limited to, stem cells, thymocytes, precursor cells and the like. A precursor cell population includes cells of a mesodermal derived cellular lineage, hematopoietic lineage, endothelial lineage, muscle cell lineage, epithelial cell lineage and neural cell lineage.

A “progenitor cell” or “stem cell” is used interchangeably herein and can be any cell in a cell differentiation pathway that is capable of differentiating into a more mature cell. As such, the term “progenitor cell population” or “stem cell population” refers to a group of cells capable of developing into a more mature cell. A stem cell population can comprise cells that are totipotent, cells that are pluripotent and cells that are stem cell lineage restricted (i.e. cells capable of developing into less than all hematopoietic lineages, or into, for example, only cells of erythroid lineage).

As used herein, the term “totipotent cell” refers to a stem cell capable of developing into all lineages of cells. Similarly, the term “totipotent population of cells” refers to a composition of cells capable of developing into all lineages of cells. Also as used herein, the term “pluripotent cell” refers to a cell capable of developing into a variety (albeit not all) lineages and are at least able to develop into all hematopoietic lineages (e.g., lymphoid, erythroid, and thrombocytic lineages). For example, a pluripotent cell can differ from a totipotent cell by having the ability to develop into all cell lineages except endothelial cells. A “pluripotent population of cells” refers to a composition of cells capable of developing into less than all lineages of cells but at least into all hematopoietic lineages. As such, a totipotent cell or composition of cells is less developed than a pluripotent cell or compositions of cells. As used herein, the terms “develop”, “differentiate” and “mature” all refer to the progression of a cell from the stage of having the potential to differentiate into at least two different cellular lineages to becoming a specialized cell. Such terms can be used interchangeably for the purposes of the present application.

As used herein, the term “population” refers to cells having the same or different identifying characteristics. The term “lineage” refers to all of the stages of the development of a cell type, from the earliest precursor cell to a completely mature cell (i.e. a specialized cell). A stem cell population can develop into cells, for example, of mesodermal cell lineage, of ectodermal cell lineage or of endodermal cell lineage. As used herein, mesodermal cells include cells of connective tissue, bone, cartilage, muscle, blood and blood vessel, lymphatic and lymphoid organ, notochord, pleura, pericardium, peritoneum, kidney and gonad. Ectodermal cells include epidermal tissue cells, such as those of nail, hair, glands of the skin, the nervous system, the external sense organs (e.g., eyes and ears) and mucous membranes (such as those of the mouth and anus). Endodermal cells include cells of the epithelium such as those of the pharynx, respiratory tract (except the nose), digestive tract, bladder and urethra cells. Cells within a stem cell population of the present invention include cells of at least one of the following cellular lineages: hematopoietic cell lineage, endothelial cell lineage, epithelial cell lineage, muscle cell lineage and neural cell lineage. Other cells within a stem cell population of the present invention include cells of erythroid lineage, endothelial lineage, leukocyte lineage, thrombocyte lineage, erythroid lineage (including primitive and definitive erythroid lineages), macrophage lineage, neutrophil lineage, mast cell lineage, megakaryocyte lineage, natural killer cell lineage, eosinophil lineage, T cell lineage, endothelial cell lineage and B cell lineage.

Various techniques may be employed to separate the cells by initially removing cells of dedicated lineage. Monoclonal antibodies are particularly useful for identifying markers associated with particular cell lineages and/or stages of differentiation.

If desired, a large proportion of terminally differentiated cells may be removed by initially using a “relatively crude” separation. For example, magnetic bead separations may be used initially to remove large numbers of lineage committed cells. Desirably, at least about 80%, usually at least 70% of the total stem cells will be removed.

Procedures for separation may include but are not limited to, magnetic separation, using antibody-coated magnetic beads, affinity chromatography, cytotoxic agents joined to a monoclonal antibody or used in conjunction with a monoclonal antibody, including but not limited to, complement and cytotoxins, and “panning” with antibody attached to a solid matrix, e.g., plate, elutriation or any other convenient technique.

Techniques providing accurate separation include but are not limited to, flow cytometry, which can have varying degrees of sophistication, e.g., a plurality of color channels, low angle and obtuse light scattering detecting channels, impedance channels, etc.

Compositions for Amplification

In a preferred embodiment, compositions and/or methods are provided for determining molecular mechanisms underlying stem cells self-renewal and differentiation into specific cell lineages that can generate different tissues. In particular, methods for determining low copy gene expression profiling of stem cells, are provided. Preferred cells of the invention are stem cells at any stage of differentiation, however, totipotent stem cells are preferred. Any cell can be used in the methods of the invention, including but not limited to, embryonic stem cells, thymocytes, precursor cells and the like. A precursor cell population includes cells of a mesodermal derived cellular lineage, hematopoietic lineage, endothelial lineage, muscle cell lineage, epithelial cell lineage and neural cell lineage.

In a preferred embodiment, a modified oligo-dT primer and T7-SWITCH primer bearing T7 RNA polymerase site is used in the first-strand synthesis reaction (FIG. 1—Flow Chart). FIG. 1 is a schematic illustration of the method described herein, and is not meant to limit or construe the invention in anyway. When reverse transcriptase reaches the 5′ end of mRNA, the enzyme's terminal transferase activity adds a few additional nucleotides, primarily deoxycitidine, to the 3′ end of the cDNA. The T7-SWITCH primer, which has an oligo (rG) sequence at its 3′ end, base-pairs with the deoxycitidine stretch, creating an extended template. Reverse transcriptase (RT) then switches templates and continues replicating to the end of the oligonucleotide. The resulting full-length, single stranded cDNA contains the complete 5′ end of the mRNA, as well as sequences that are complimentary to the T7-SWITCH oligonucleotide. The T7-SWITCH anchor sequence and the polyA sequence serve as universal priming sites for end-to-end cDNA amplification via long-distance PCR using only one specific primer. The dsDNA contains a T7 RNA Polymerase site which is used for the production of sense RNA during T7 Polymerase amplification step. The resultant sense RNA can be a subject of the second round of the amplification (FIG. 1—Flow Chart).

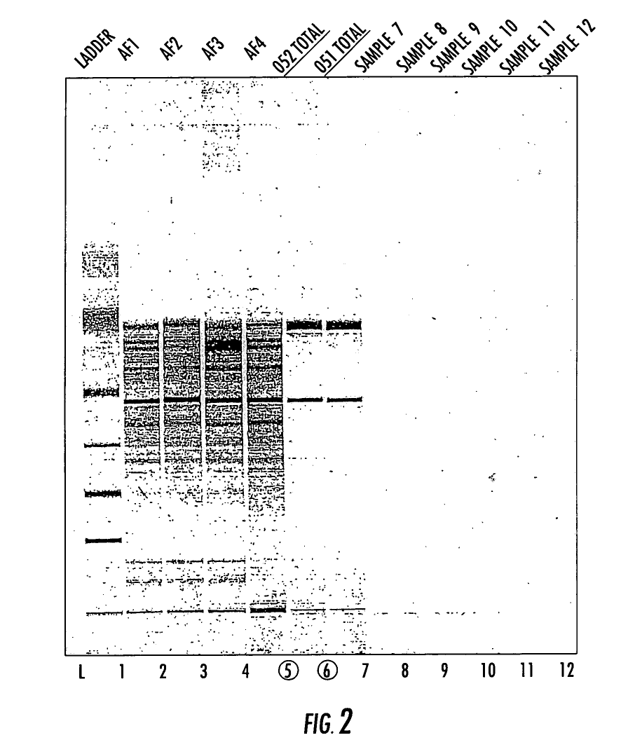

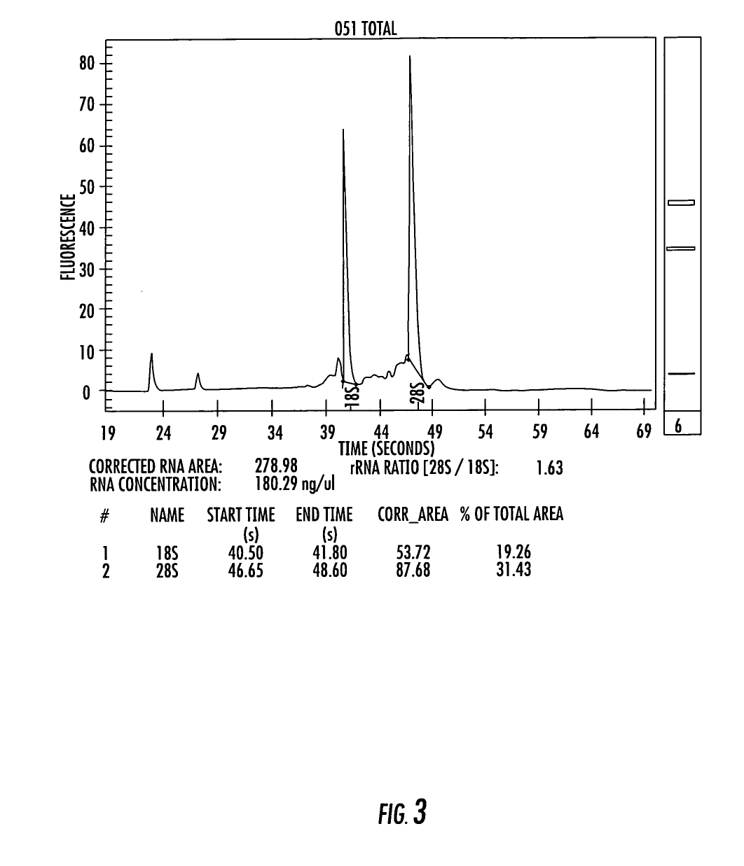

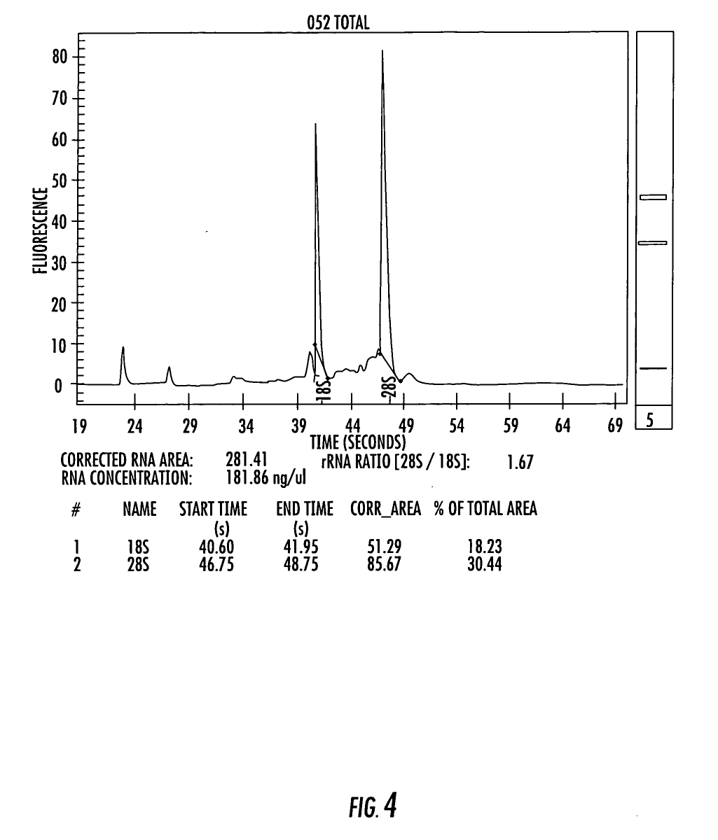

In another preferred embodiment, the amplified product is characterized by comparing the different lengths, structures and the abundance of specific transcripts. For example, candidate genes are chosen to represent all groups of abundance designated herein as: 1) abundant (approximately 1,000-3,000 copies per cell); 2) moderate expressed genes (300-1,000 copies per cell); 3) rare genes (less than 300 copies per cell). Each group comprises transcripts which differ in the length (small, medium, long). The human glioma LN 229 cell line has been chosen as a test sample. Total RNA was isolated from cells growing on six plates (50-70% of confluent), which provides enough material to compare the amplified and the original sample. The LN 229 population was propagated from a single cell. The quality of isolated total RNA was confirmed using an Agilent 2100 Bioanalyzer (FIG. 2-4, samples 051, 052-total). Four transcripts were selected to characterize the samples with or without a round(s) of amplification using real-time PCR. Two of them belong to the group of abundant genes: actin (the length is 1,800 nt; 3,200 copies per cell) and tenascin (the length is 7,500 nt; 1,100 copies per cell). Other candidates are rare genes: TBP (the length is 1,900 nt; 76 copies per cell) and TFRC (the length is 5,000 nt, 160 copies per cell). The approximate copy number was calculated based on the calibration curve of each given transcript. To achieve this, full-length transcripts were amplified by PCR using proof-reading DNA Polymerase. This approach allows monitoring 5′/3′ ratio of amplified products. To further describe this amplification system, more markers (up to nine) from the aforementioned groups are applied.

The different amounts of total RNA from the LN 229 cell line, as well from single glioma-derived “neurospheres”, were taken for the amplification procedure: 0.5 ng, 1 ng, 2 ng, 5 ng, 20 ng. A modified oligo-dT primer and T7-SWITCH primer bearing T7 RNA polymerase site were used in the first-strand synthesis reaction (FIG. 1—Flow Chart). When reverse transcriptase reaches the 5′ end of mRNA, the enzyme's terminal transferase activity adds a few additional nucleotides, primarily deoxycitidine, to the 3′ end of the cDNA. The T7-SWITCH primer, which has an oligo (rG) sequence at its 3′ end, base-pairs with the deoxycitidine stretch, creating an extended template. RT then switches templates and continues replicating to the end of the oligonucleotide. The resulting full-length, single stranded cDNA contains the complete 5′ end of the mRNA, as well as sequences that are complimentary to the T7-SWITCH oligonucleotide. The T7-SWITCH anchor sequence and the polyA sequence serve as universal priming sites for end-to-end cDNA amplification via long-distance PCR using only one specific primer. The dsDNA contains a T7 RNA Polymerase site which is used for the production of sense RNA during T7 Polymerase amplification step. The resultant sense RNA can be a subject of the second round of the amplification (FIG. 1—Flow Chart). FIG. 1 is a schematic illustration of the method described herein.

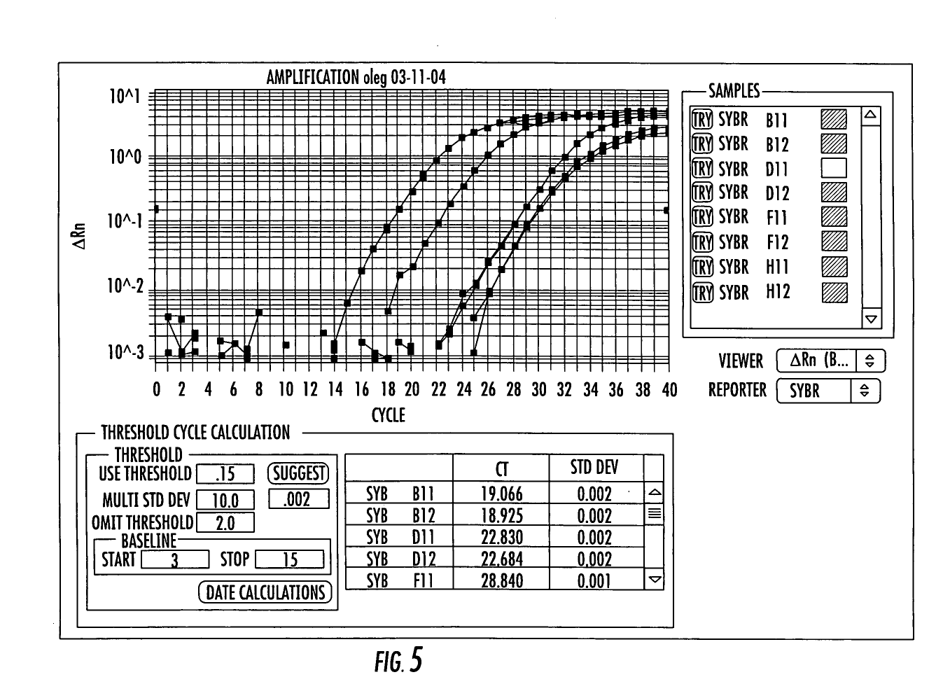

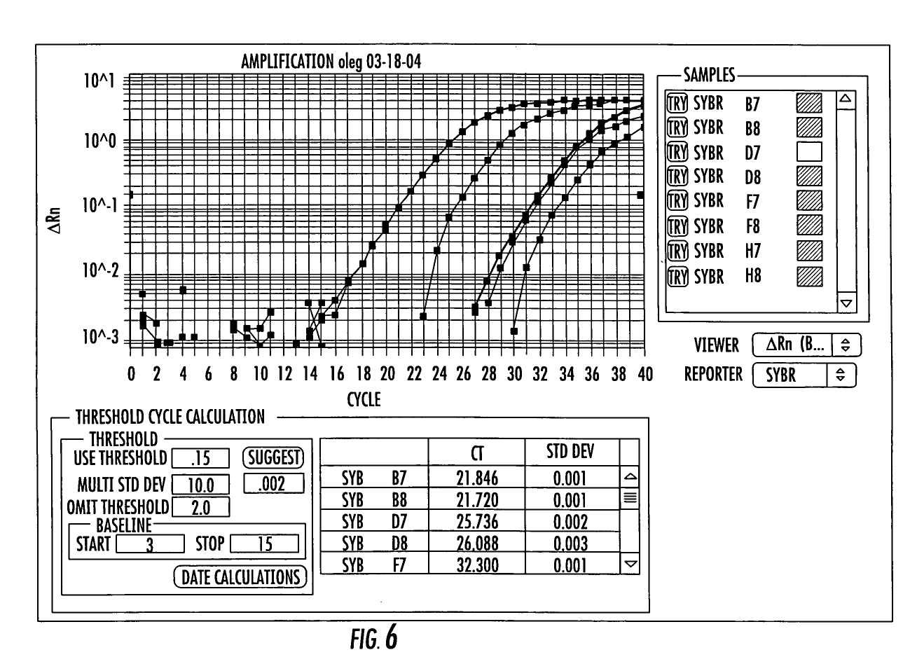

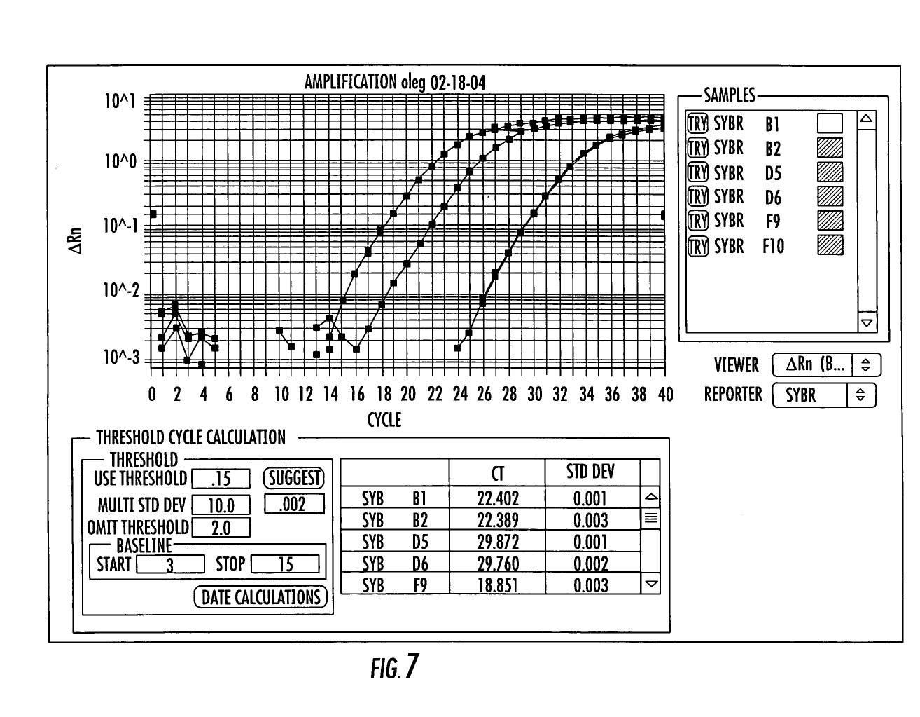

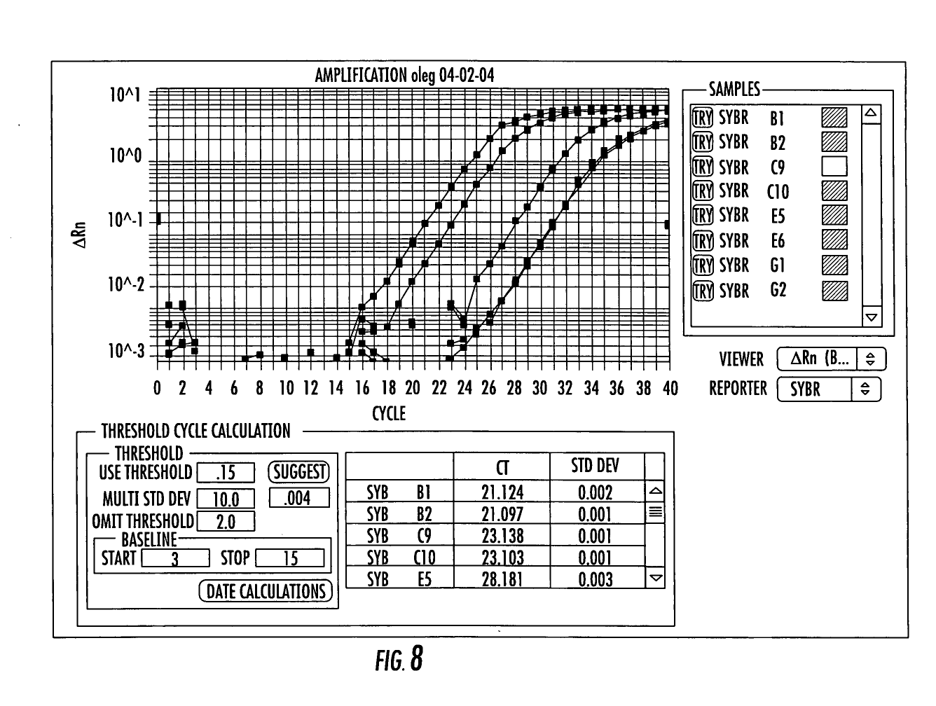

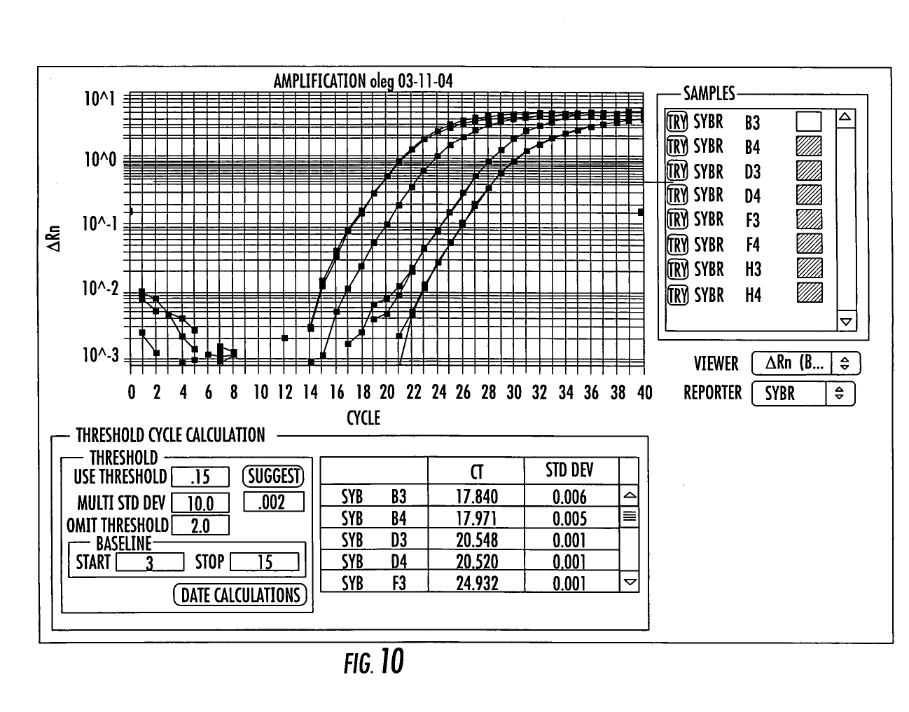

The expression profile of different amounts of total RNA from LN 229 (0.5-20 ng range) that are amplified resembles that of the sample without amplification (FIGS. 5-10). Total RNA (1,860 ng) of the original sample was taken for RT without amplification round(s). To mimic the isolation of RNA from small numbers of cells, 1 ng of total RNA from LN 229 was taken through the RNA isolation process again. The isolation of nucleic acids from a few cells led to a dramatic loss of material (up to 50-75%). The approximately 250 pg of total RNA after isolation was split into two tubes for further amplification. This amount of total RNA corresponds to 4-6 cells—if one cell contains 20-30 pg of total RNA, 5 cells contain approximately 125 pg.

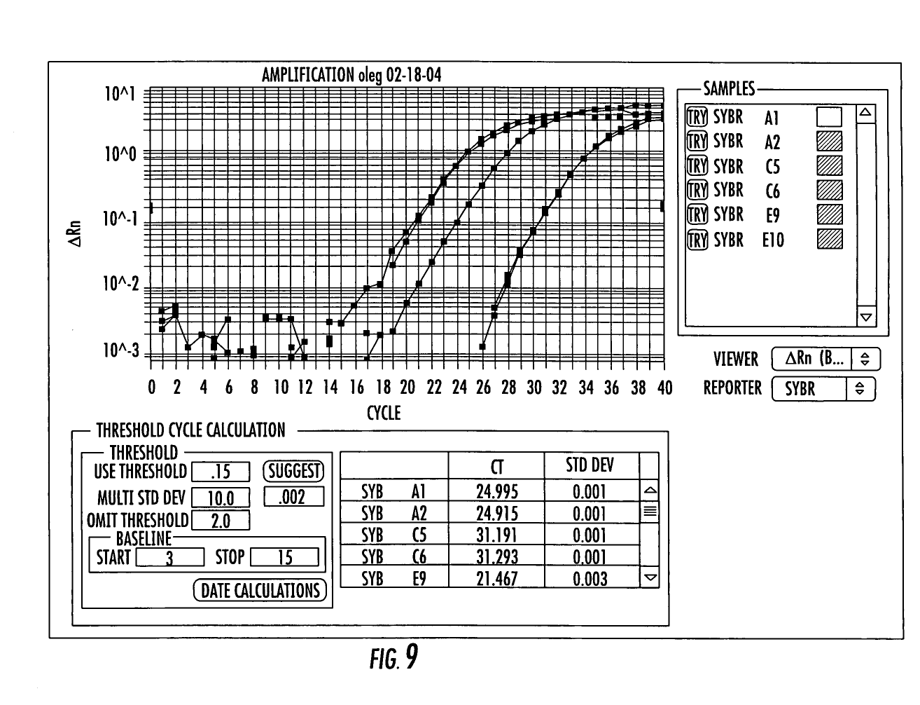

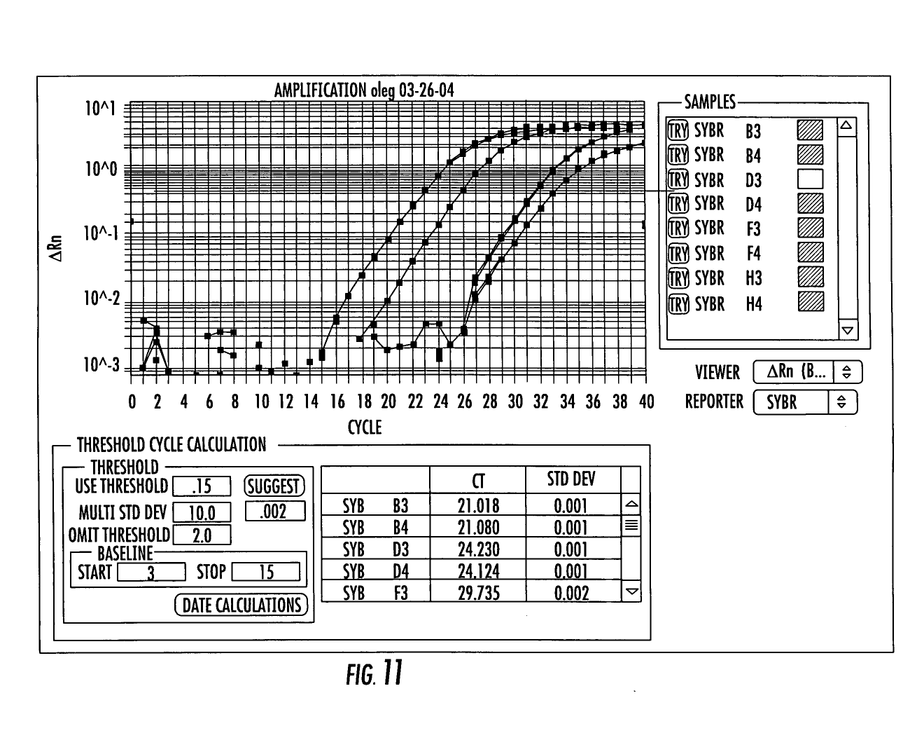

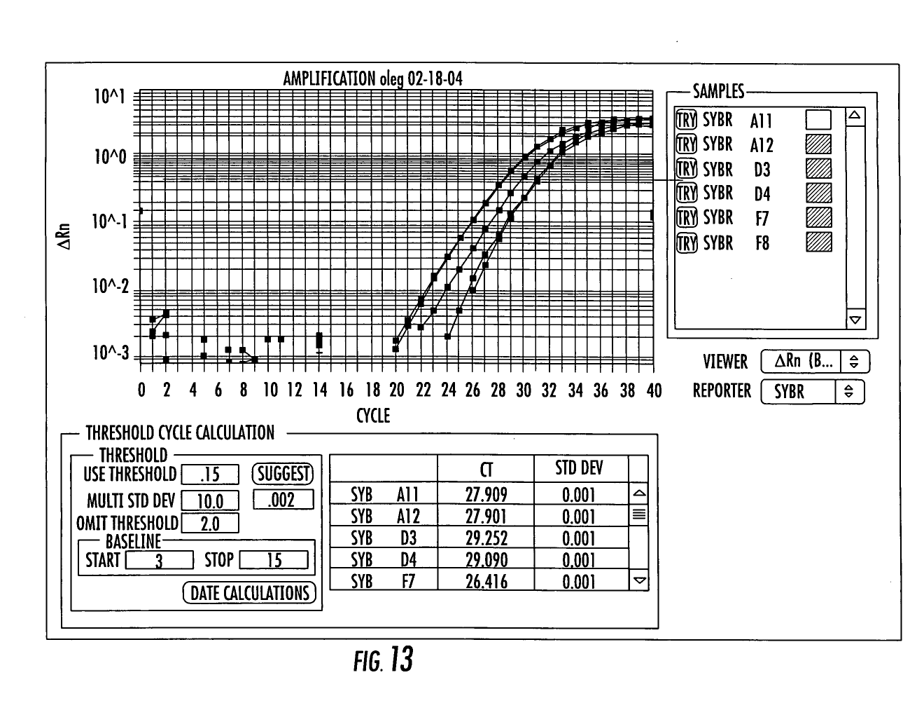

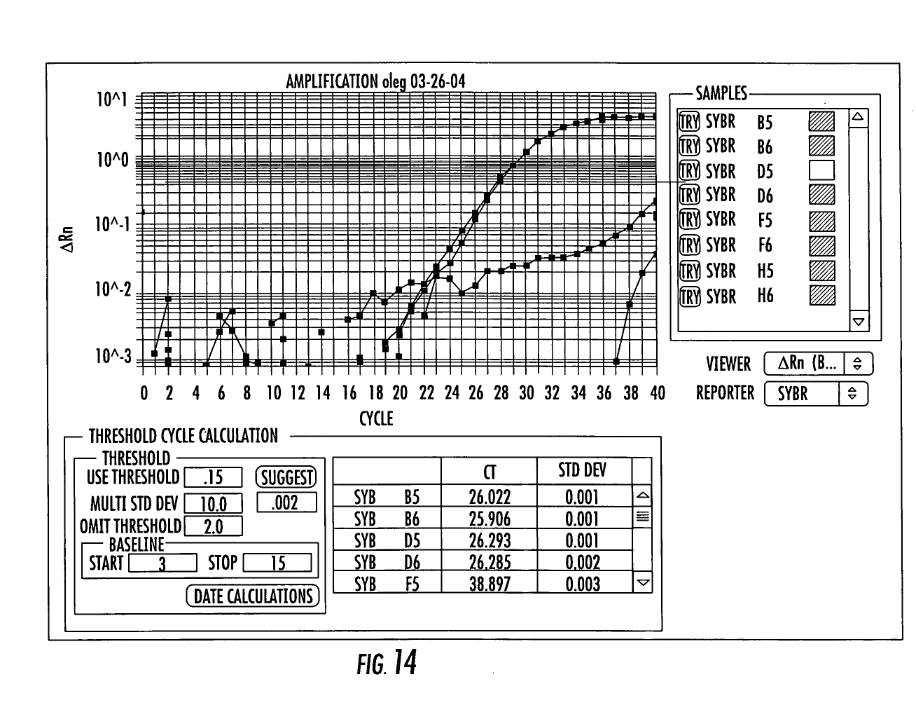

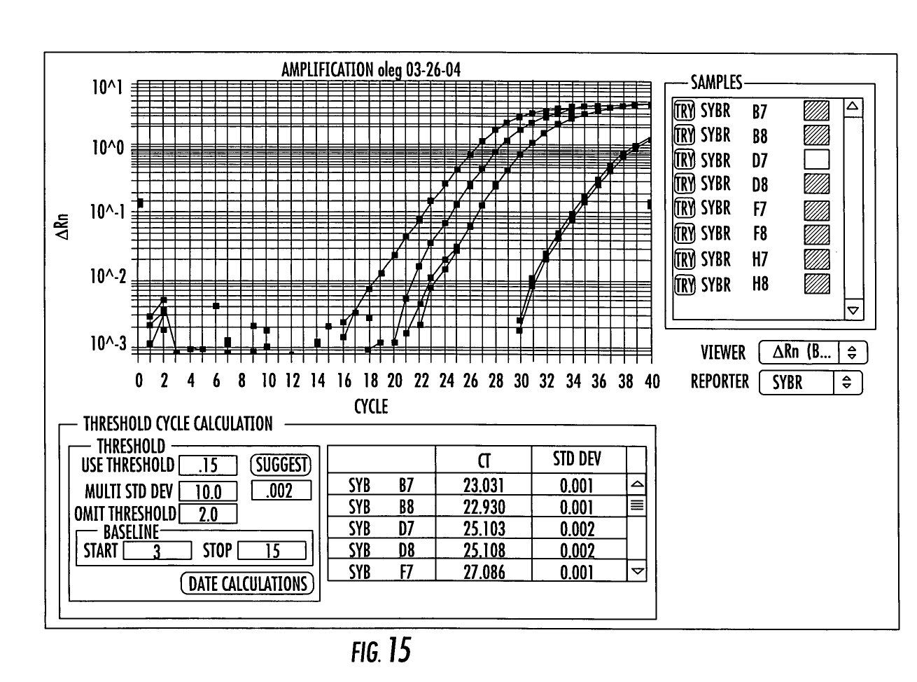

A comparison of one and two rounds of amplification yields similar amplification results (FIGS. 9 and 11, 12 and 14, 13 and 15) that can be explained by the similarity of amplification approaches and the template's identity (sense RNA) used in the first and the second rounds. In contrast, the aRNA amplification methods, classic and modified, utilize different primers in every round and different types of template: sense—for the first round, antisense—for the second.

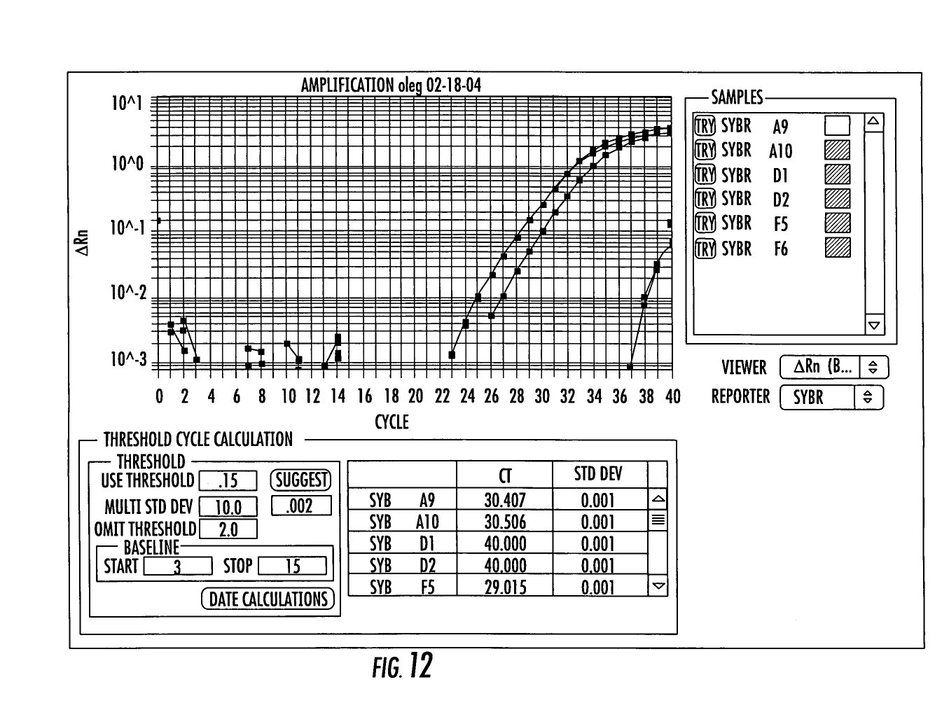

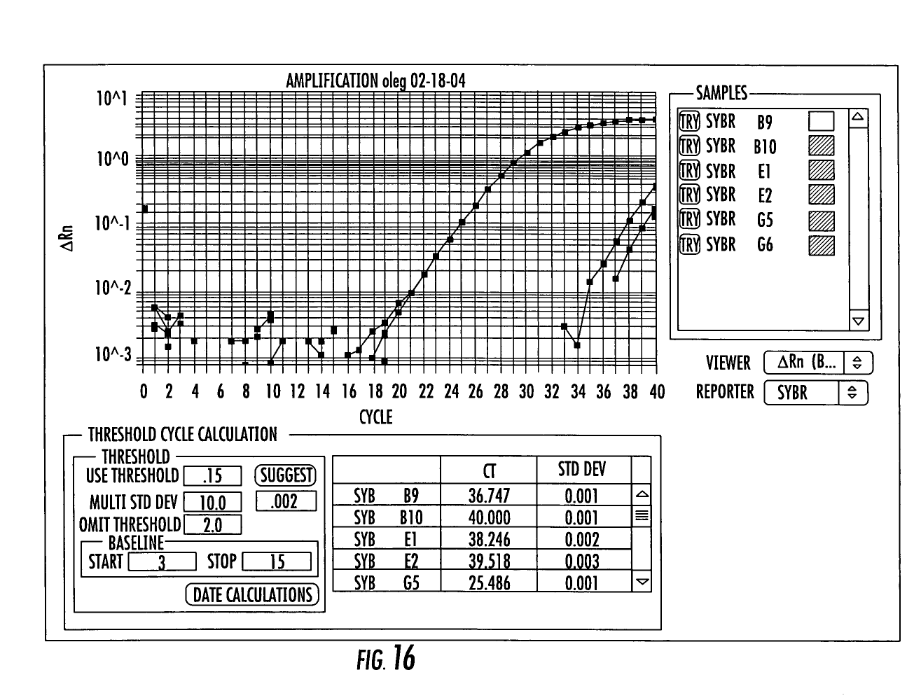

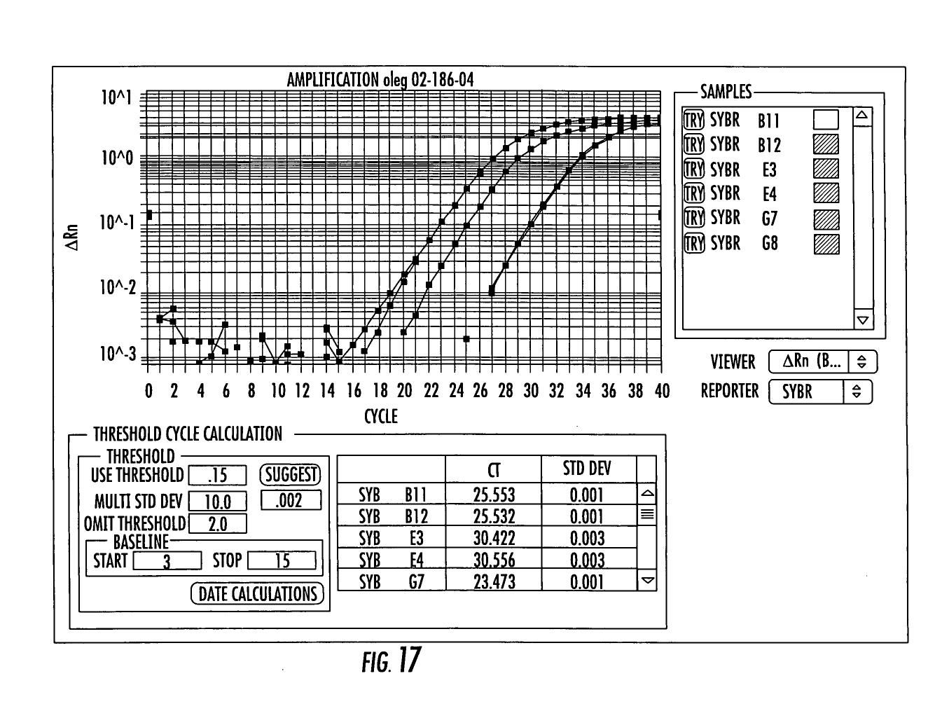

Optimum numbers of cycles were determined. A large distortion was shown when the amplification products from single glioma spheres were obtained using 21 cycles of PCR as compared to 12 cycles (FIGS. 12 and 16, 13 and 17).

The present method utilizes: 5′ end enrichment, and limited amplification by a SMART-like method that prevents template loss when compared to T7 RNA amplification, providing a high template-yield. The linearity of amplification by DNA-dependent RNA Polymerase and its processivity provide the conservation of a minimal sample's distortion introduced by PCR. Methods known in the art require higher amounts of genetic material and produce lower yields. A method has recently been published (Rajeevan, et al., Genomics, 2003) where the investigators used 900-1000 ng of total RNA, yielding 2×10³-2.5×10³ fold amplification in comparison with 0.5 ng (up to 10⁵ fold) for the present system. Furthermore, 18 cycles of PCR utilized by other investigators during the long-distance step could introduce additional sample distortion, especially for rare transcripts. Moreover two different primers were used during PCR that may contribute to non-specific amplification and decrease the efficiency of long-distance PCR. In contrary, the system described here utilizes only one amplification primer.

Advantages of Disclosed Amplification Method

There are particular advantages of this novel technology for RNA amplification:

The dsDNA pool can be used as a biorepository since one may use only a fraction of it for further amplification(s).

The orientation of T7 RNA Polymerase site, upstream of ssDNA, provides a production of sense RNA; as a result the second round of amplification can be made in the same manner as a first round (FIG. 1—Flow chart).

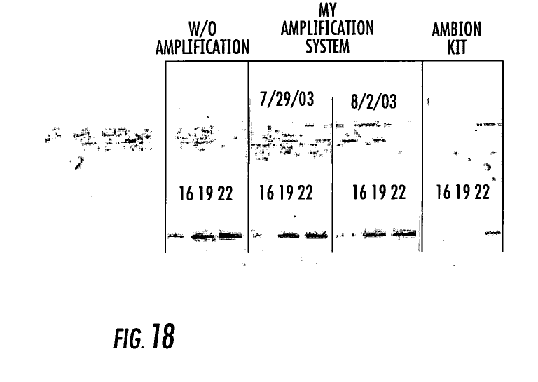

The amplified population of transcripts is longer in size and 5′ end-enriched, compared to the modified Eberwine method (Ambion kit) (FIG. 18).

When using an array, the first strand DNA could be introduced instead of antisense RNA, addressing the RNA degradation concern.

High Density Arrays

In a preferred embodiment, amplification libraries generated by the methods disclosed herein, can be used for array screening and the information derived from such a library may be utilized during the array production. High density arrays are particularly useful for monitoring the expression control at the transcriptional, RNA processing and degradation level. The fabrication and application of high density arrays in gene expression monitoring have been disclosed previously in, for example, WO 97/10365, WO 92/10588, U.S. application Ser. No. 08/772,376 filed Dec. 23, 1996; Ser. No. 08/529,115 filed on Sep. 15, 1995; Ser. No. 08/168,904 filed Dec. 15, 1993; Ser. No. 07/624,114 filed on Dec. 6, 1990, Ser. No. 07/362,901 filed Jun. 7, 1990, all incorporated herein for all purposed by reference. In some embodiment using high density arrays, high density oligonucleotide arrays are synthesized using methods such as the Very Large Scale Immobilized Polymer Synthesis (VLSIPS) disclosed in U.S. Pat. No. 5,445,934 incorporated herein for all purposes by reference. Each oligonucleotide occupies a known location on a substrate. A nucleic acid target sample is hybridized with a high density array of oligonucleotides and then the amount of target nucleic acids hybridized to each probe in the array is quantified. One preferred quantifying method is to use confocal microscope and fluorescent labels. The GeneChip™ system (Affymetrix, Santa Clara, Calif.) is particularly suitable for quantifying the hybridization; however, it will be apparent to those of skill in the art that any similar systems or other effectively equivalent detection methods can also be used.

High density arrays are suitable for quantifying a small variations in expression levels of a gene in the presence of a large population of heterogeneous nucleic acids. Such high density arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. Suitable nucleic acids are also produced by amplification of templates. As a nonlimiting illustration, polymerase chain reaction, and/or in vitro transcription, are suitable nucleic acid amplification methods, however, the preferred method is PCR.

Synthesized oligonucleotide arrays are particularly preferred for this invention. Oligonucleotide arrays have numerous advantages, as opposed to other methods, such as efficiency of production, reduced intra- and inter array variability, increased information content and high signal-to-noise ratio.

Preferred high density arrays for gene function identification and genetic network mapping comprise greater than about 100, preferably greater than about 1000, more preferably greater than about 16,000 and most preferably greater than 65,000 or 250,000 or even greater than about 1,000,000 different oligonucleotide probes, preferably in less than 1 cm² of surface area. The oligonucleotide probes range from about 5 to about 50 or about 500 nucleotides, more preferably from about 10 to about 40 nucleotide and most preferably from about 15 to about 40 nucleotides in length.

One of skill in the art would appreciate that in order to interrogate the genetic network, it is desirable to measure the control of transcription. Because all the cell nuclei of an organism generally carry the same genes, the difference in the protein products in different cell types is generally the result of selective gene expression. It is well known in the art that the first level of regulation is at the level of transcription, i.e., by varying the frequency with which a gene is transcribed into nascent pre-mRNA by a RNA polymerase. The regulation of transcription is one of the most important steps in the control of gene expression because transcription constitutes the input of the mRNA pool. It is generally known in the art that transcriptional regulation can be achieved through various means. As non-limiting examples, transcription can be controlled by a) cis-acting transcriptional control sequences and transcriptional factors; b) different gene products from a single transcription unit and c) epigenetic mechanisms; and d) long range control of genetic expression by chromatin structure. The current invention provides methods for detecting the transcriptional regulation of individual genes at all of these levels of control.

Primers as Capture Probes on Microarrays

In a preferred embodiment, amplified nucleic acid molecules from samples using the methods and/or compositions described herein, can be identified by immobilizing primers on substrate surfaces, such as a microarray. Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

Assays using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

The capture probes or oligonucleotides used in the methods of the present invention may be used without any prior analysis of the structure assumed by a target nucleic acid. For any given case, it can be determined empirically using appropriately selected reference target molecules whether a chosen probe or array of probes can distinguish between genetic variants sufficiently for the needs of a particular assay. Once a probe or array of probes is selected, the analysis of which probes bind to a target, and how efficiently these probes bind (i.e., how much of probe/target complex can be detected) allows a hybridization signature of the conformation of the target to be created. It is contemplated that the signature may be stored, represented or analyzed by any of the methods commonly used for the presentation of mathematical and physical information, including but not limited to line, pie, or area graphs or 3-dimensional topographic representations. The data may also be used as a numerical matrix, or any other format that may be analyzed either visually, mathematically or by computer-assisted algorithms, such as for example nucleic acid analysis software.

The resulting signatures of the nucleic acid structures serve as sequence-specific identifiers of the particular molecule, without requiring the determination of the actual nucleotide sequence. While specific sequences may be identified by comparison of their signature to a reference signature, the use of algorithms to deduce the actual sequence of a molecule by sequence-specific hybridization (i.e., at high stringency to eliminate the influence of secondary and tertiary structures) to a complete matrix (i.e., probes that shift by a single nucleotide position at each location of an array), is not a feature or requirement, or within the bounds of the methods of the present invention.

It is also contemplated that information on the structures assumed by a target nucleic acid may be used in the design of the probes, such that regions that are known or suspected to be involved in folding may be chosen as hybridization sites. Such an approach will reduce the number of probes that are likely to be needed to distinguish between targets of interest.

There are many methods used to obtain structural information involving nucleic acids, including the use of chemicals that are sensitive to the nucleic acid structure, such as phenanthroline/copper, EDTA-Fe²⁺, cisplatin, ethylnitrosourea, dimethylpyrocarbonate, hydrazine, dimethyl sulfate, and bisulfite. Enzymatic probing using structure-specific nucleases from a variety of sources, such as the Cleavase™ enzymes (Third Wave Technologies, Inc., Madison, Wis.), Taq DNA polymerase, E. coli DNA polymerase I, and eukaryotic structure-specific endonucleases (e.g., human, murine and Xenopus XPG enzymes, yeast RAD2 enzymes), murine FEN-1 endonucleases (Harrington and Lieber, Genes and Develop., 3:1344 [1994]) and calf thymus 5′ to 3′ exonuclease (Murante et al., J. Biol. Chem., 269:1191 [1994]). In addition, enzymes having 3′ nuclease activity such as members of the family of DNA repair endonucleases (e.g., the RrpI enzyme from Drosophila melanogaster, the yeast RAD1/RAD10 complex and E. coli Exo III), are also suitable for examining the structures of nucleic acids.

If analysis of structure as a step in probe selection is to be used for a segment of nucleic acid for which no information is available concerning regions likely to form secondary structures, the sites of structure-induced modification or cleavage must be identified. It is most convenient if the modification or cleavage can be done under partially reactive conditions (i.e., such that in the population of molecules in a test sample, each individual will receive only one or a few cuts or modifications). When the sample is analyzed as a whole, each reactive site should be represented, and all the sites may be thus identified. Using a Cleavase Fragment Length Polymorphism™ cleavage reaction as an example, when the partial cleavage products of an end labeled nucleic acid fragment are resolved by size (e.g., by electrophoresis), the result is a ladder of bands indicating the site of each cleavage, measured from the labeled end. Similar analysis can be done for chemical modifications that block DNA synthesis; extension of a primer on molecules that have been partially modified will yield a nested set of termination products. Determining the sites of cleavage/modification may be done with some degree of accuracy by comparing the products to size markers (e.g., commercially available fragments of DNA for size comparison) but a more accurate measure is to create a DNA sequencing ladder for the same segment of nucleic acid to resolve alongside the test sample. This allows rapid identification of the precise site of cleavage or modification.

The oligonucleotides or capture probe may interact with the target in any number of ways. For example, in another embodiment, the capture probes may contact more than one region of the target nucleic acid. When the target nucleic acid is folded, two or more of the regions that remain single stranded may be sufficiently proximal to allow contact with a single capture probe. The capture oligonucleotide in such a configuration is referred to herein as a “bridge” or “bridging” oligonucleotide, to reflect the fact that it may interact with distal regions within the target nucleic acid. The use of the terms “bridge” and “bridging” is not intended to limit these distal interactions to any particular type of interaction. It is contemplated that these interactions may include non-standard nucleic acid interactions known in the art, such as G-T base pairs, Hoögsteen interactions, triplex structures, quadraplex aggregates, and the multibase hydrogen bonding such as is observed within nucleic acid tertiary structures, such as those found in tRNA's. The terms are also not intended to indicate any particular spatial orientation of the regions of interaction on the target strand, i.e., it is not intended that the order of the contact regions in a bridge oligonucleotide be required to be in the same sequential order as the corresponding contact regions in the target strand. The order may be inverted or otherwise shuffled.

Identification of Nucleic Acid Sequences In Vivo.

With respect to the cloning of allelic variants of the mammalian genes such as human, and homologues from other species (e.g., mouse), isolated gene sequences of interest may be labeled and used to screen a cDNA library constructed from mRNA obtained from cells or tissues (e.g., stem cells, brain tissues) derived from the organism (e.g., mouse) of interest. The hybridization conditions used should be of a lower stringency when the cDNA library is derived from an organism different from the type of organism from which the labeled sequence was derived.

Alternatively, the labeled fragment may be used to screen a genomic library derived from the organism of interest, again, using appropriately stringent conditions, as described in detail the Examples which follow. Low stringency conditions are well known to those of skill in the art, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Press, N.Y.; and Ausubel, et al., 1989, Current Protocols in Molecular Biology, Green Publishing Associates and Wiley Interscience, N.Y.

Further, a gene allelic variant may be isolated from, for example, human nucleic acid, by performing the PCR methods of the invention. For example, the template for the reaction may be cDNA obtained by reverse transcription of mRNA prepared from, for example, human or non-human cell lines or tissue.

A preferred method of PCR amplification using, for example a modified oligo-dT primer and T7-SWITCH primer bearing T7 RNA polymerase site in the first-strand synthesis reaction (FIG. 1—Flow Chart). When reverse transcriptase reaches the 5′ end of mRNA, the enzyme's terminal transferase activity adds a few additional nucleotides, primarily deoxycitidine, to the 3′ end of the cDNA. The T7-SWITCH primer, preferably comprises an oligo (rG) sequence at its 3′ end, base-pairs with a deoxycitidine stretch, creating an extended template. RT then switches templates and continues replicating to the end of the oligonucleotide. The resulting full-length, single stranded cDNA comprises the complete 5′ end of the mRNA, as well as sequences that are complimentary to the T7-SWITCH oligonucleotide. The T7-SWITCH anchor sequence and the polyA sequence serve as universal priming sites for end-to-end cDNA amplification via long-distance PCR using only one specific primer. The dsDNA comprises a T7 RNA Polymerase site which is used for the production of sense RNA during T7 Polymerase amplification step. The resultant sense RNA can be a subject of the second round of the amplification (FIG. 1—Flow Chart). This method is also used to determine the absence of any gene expression when comparing for example, normal cells and cells from an individual suffering from or susceptible to any disorder, such as for example neuronal disorders.

The PCR technology disclosed herein, can also be utilized to isolate full length cDNA sequences. For example, RNA may be isolated, following standard procedures, from a cellular or tissue source. For example, one known, or suspected, to express a neural developmental gene, such as, for example, brain tissue samples obtained through biopsy or post-mortem, stem cells and the like). A reverse transcription reaction may be performed on the RNA using an oligonucleotide primer as described infra. The resulting RNA/DNA hybrid may then be “tailed” with guanines using a standard terminal transferase reaction, the hybrid may be digested with RNAse H, and second strand synthesis may then be primed with a poly-C primer. Thus, cDNA sequences upstream of the amplified fragment may easily be isolated. For a review of cloning strategies that may be used, see e.g., Sambrook et al., 1989, infra.

Another preferred method includes combining the PCR methods and compositions described infra, with SAGE. Serial Analysis of Gene Expression (SAGE), is based on the identification of and characterization of partial, defined sequences of transcripts corresponding to gene segments. These defined transcript sequence “tags” are markers for genes which are expressed in a cell, a tissue, or an extract, for example.

SAGE is based on several principles. First, a short nucleotide sequence tag (9 to 10 bp) contains sufficient information content to uniquely identify a transcript provided it is isolated from a defined position within the transcript. For example, a sequence as short as 9 bp can distinguish about 262,144 transcripts given a random nucleotide distribution at the tag site, whereas estimates suggest that the human genome encodes about 80,000 to 200,000 transcripts (Fields, et al., Nature Genetics, 7:345 1994). The size of the tag can be shorter for lower eukaryotes or prokaryotes, for example, where the number of transcripts encoded by the genome is lower. For example, a tag as short as 6-7 bp may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. The concept of deriving a defined tag from a sequence in accordance with the present invention is useful in matching tags of samples to a sequence database. In the preferred embodiment, a computer method is used to match a sample sequence with known sequences.

The tags used herein, uniquely identify genes. This is due to their length, and their specific location (3′) in a gene from which they are drawn. The full length genes can be identified by matching the tag to a gene data base member, or by using the tag sequences as probes to physically isolate previously unidentified genes from cDNA libraries. The methods by which genes are isolated from libraries using DNA probes are well known in the art. See, for example, Veculescu et al., Science 270: 484 (1995), and Sambrook et al. (1989), MOLECULAR CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above method but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

In another preferred embodiment, Expressed Sequenced Tags (ESTs), can also be used to identify nucleic acid molecules which are over expressed in a neuronal cell. ESTs from a variety of databases can be identified. For example, preferred databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet, at, for example, ncbi.nlm.nih.gov/Omim/. CGAP, which is an interdisciplinary program to establish the information and technological tools required to decipher the molecular anatomy of a cancer cell. CGAP can be accessed through the world wide web of the Internet, at, for example, ncbi.nlm.nih.gov/ncicgap/. Some of these databases may contain complete or partial nucleotide sequences. In addition, alternative transcript forms can also be selected from private genetic databases. Alternatively, nucleic acid molecules can be selected from available publications or can be determined especially for use in connection with the present invention.

Alternative transcript forms can be generated from individual ESTs which are within each of the databases by computer software which generates contiguous sequences. In another embodiment of the present invention, the nucleotide sequence of the nucleic acid molecule is determined by assembling a plurality of overlapping ESTs. The EST database (dbEST), which is known and available to those skilled in the art, comprises approximately one million different human mRNA sequences comprising from about 500 to 1000 nucleotides, and various numbers of ESTs from a number of different organisms. dbEST can be accessed through the world wide web of the Internet, at, for example, ncbi.nlm.nih.gov/dbEST/index.html. These sequences are derived from a cloning strategy that uses cDNA expression clones for genome sequencing. ESTs have applications in the discovery of new genes, mapping of genomes, and identification of coding regions in genomic sequences. Another important feature of EST sequence information that is becoming rapidly available is tissue-specific gene expression data. This can be extremely useful in targeting selective gene(s) for therapeutic intervention. Since EST sequences are relatively short, they must be assembled in order to provide a complete sequence. Because every available clone is sequenced, it results in a number of overlapping regions being reported in the database. The end result is the elicitation of alternative transcript forms from, for example, immune cells and neuronal cells.

Assembly of overlapping ESTs extended along both the 5′ and 3′ directions results in a full-length “virtual transcript.” The resultant virtual transcript may represent an already characterized nucleic acid or may be a novel nucleic acid with no known biological function. The Institute for Genomic Research (TIGR) Human Genome Index (HGI) database, which is known and available to those skilled in the art, contains a list of human transcripts. TIGR can be accessed through the world wide web of the Internet, at, for example, tigr.org. Transcripts can be generated in this manner using TIGR-Assembler, an engine to build virtual transcripts and which is known and available to those skilled in the art. TIGR-Assembler is a tool for assembling large sets of overlapping sequence data such as ESTs, BACs, or small genomes, and can be used to assemble eukaryotic or prokaryotic sequences. TIGR-Assembler is described in, for example, Sutton, et al., Genome Science & Tech., 1995, 1, 9-19, which is incorporated herein by reference in its entirety, and can be accessed through the file transfer program of the Internet, at, for example, tigr.org/pub/software/TIGR. assembler. In addition, GLAXO-MRC, which is known and available to those skilled in the art, is another protocol for constructing virtual transcripts. PHRAP is used for sequence assembly within Find Neighbors and Assemble EST Blast. PHRAP can be accessed through the world wide web of the Internet, at, for example, chimera.biotech.washington.edu/uwgc/tools/phrap.htm. Identification of ESTs and generation of contiguous ESTs to form full length RNA molecules is described in detail in U.S. application Ser. No. 09/076,440, which is incorporated herein by reference in its entirety.

In another preferred embodiment, alternative transcript information could be also retrieved from other gene databases, such as for example, LOCUSLINK, Alternative Splicing Database (ASD), and ASAP database.

Modified Primers

In another preferred embodiment, primers used in the PCR disclosed herein can comprise modified nucleobases.

The term “succeeding monomer” relates to the neighboring monomer in the 5′-terminal direction and the “preceding monomer” relates to the neighboring monomer in the 3′-terminal direction.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, inosine with C, pseudoisocytosine with G, etc.

Preferred oligonucleotides of the invention also may have at least one non-modified nucleic acid located either at or within a distance of no more than three bases from the mismatch position(s) of a complementary oligonucleotide, such as at a distance of two bases from the mismatch position, e.g. at a distance of one base from the mismatch position, e.g. at the mismatch position.

The oligonucleotides of the present invention are highly suitable for a variety of diagnostic purposes such as for the isolation, purification, amplification, detection, identification, quantification, or capture of nucleic acids such as DNA, mRNA or non-protein coding cellular RNAs, such as tRNA, rRNA, snRNA and scRNA, or synthetic nucleic acids, in vivo or in vitro.

In accordance with the invention, any desired primer may be used. For example, primers for use in the disclosed amplification method can be oligonucleotides having sequence complementary to the target sequence. This sequence is referred to as the complementary portion of the primer. The complementary portion of a primer can be any length that supports specific and stable hybridization between the primer and the target sequence under the reaction conditions. Generally, for reactions at 37° C., this can be, for example about 5 to about 35 nucleotides long or about 16 to about 24 nucleotides long. If whole genome amplification is desired, the primers can be from about 5 to about 60 nucleotides long, and in particular, can be about 5, 6, 7, 8, 9, 10, 11, 12, 13, 14, 15, 16, 17, 18, 19, and/or 20 nucleotides long.

In a preferred embodiment, target sequences that are to be amplified are of unknown sequence. For example, nucleic acid isolated from a sample for which the sequence is from an individual or any organism. In such cases, primers may be random, or of degenerate sequence (that is, use of a collection of primers having a variety of sequences), primer hybridization need not be specific. In such cases the primers need only be effective in priming synthesis. For example, in whole genome amplification specificity of priming is not essential since the goal generally is to amplify all sequences equally. Sets of random or degenerate primers can comprise primers of about 5, 6, 7, 8, 9, 10, 11, 12, 13, 14, 15, 16, 17, 18, 19, and/or 20 nucleotides long or more. Primers six nucleotides long are referred to as hexamer primers. For example, preferred primers for whole genome amplification are random hexamer primers. That is, random hexamer primers where every possible six nucleotide sequence is represented in the set of primers. Similarly, sets of random primers of other particular lengths, or of a mixture of lengths preferably comprise every possible sequence the length of the primer, or, in particular, the length of the complementary portion of the primer. Use of random primers is described in U.S. Pat. Nos. 5,043,272 and 6,214,587 the contents of which are hereby incorporated by reference in their entirety.

In another preferred embodiment, the disclosed primers, as described in the Examples which follow, can have one or more modified nucleotides. Such primers are referred to herein as modified primers. Modified primers have several advantages. First, some forms of modified primers, such as RNA/2′-O-methyl RNA chimeric primers, have a higher melting temperature (Tm) than DNA primers. This increases the stability of primer hybridization and will increase strand invasion by the primers. This will lead to more efficient priming. Also, since the primers are made of RNA, they will be exonuclease resistant. Such primers, if tagged with minor groove binders at their 5′ end, will also have better strand invasion of the template dsDNA. In addition, RNA primers can also be very useful for amplification of nucleic acid molecules from biological samples such as cells or tissue. Since the biological samples contain endogenous RNA, this RNA can be degraded with RNase to generate a pool of random oligomers, which can then be used to prime the polymerase for amplification of the DNA. This eliminates any need to add primers to the reaction. Alternatively, DNase digestion of biological samples can generate a pool of DNA oligonucleotide primers for RNA dependent DNA amplification.

Chimeric primers can also be used. Chimeric primers are primers having at least two types of nucleotides, such as both deoxyribonucleotides and ribonucleotides, ribonucleotides and modified nucleotides, or two different types of modified nucleotides. One form of chimeric primer is peptide nucleic acid/nucleic acid primers (PNA/NAP). For example, 5′-PNA-DNA-3′ or 5′-PNA-RNA-3′ primers may be used for more efficient strand invasion and polymerization invasion. The DNA and RNA portions of such primers can have random or degenerate sequences. Other forms of chimeric primers are, for example, 5′-(2′-O-Methyl)RNA-RNA-3′ or 5′-(2′-O-Methyl)RNA-DNA-3′.

Many modified nucleotides (nucleotide analogs) are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to locked nucleic acids (LNA), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Primers composed, either in whole or in part, of nucleotides with universal bases are useful for reducing or eliminating amplification bias against repeated sequences in a target sample. This would be useful, for example, where a loss of sequence complexity in the amplified products is undesirable. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can comprise inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only comprise a single modification, but may also comprise multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to complementary nucleic acids in a Watson-Crick or Hoögsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Primers can comprise nucleotides and can be made up of different types of nucleotides or the same type of nucleotides. For example, one or more of the nucleotides in a primer can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. The nucleotides can comprise bases (that is, the base portion of the nucleotide) and can (and normally will) comprise different types of bases. For example, one or more of the bases can be universal bases, such as 3-nitropyrrole or 5-nitroindole; about 10% to about 50% of the bases can be universal bases; about 50% or more of the bases can be universal bases; or all of the bases can be universal bases.

In another preferred embodiment, primers with complementary sequences to target nucleic acids are preferred. Primers may also comprise additional sequence at the 5′ end of the primer that is not complementary to the target sequence. This sequence is referred to as the non-complementary portion of the primer. The non-complementary portion of the primer, if present, serves to facilitate strand displacement during DNA replication. The non-complementary portion of the primer can also include a functional sequence such as a promoter for an RNA polymerase. The non-complementary portion of a primer may be any length, but is generally about 1 to 100 nucleotides long, and preferably about 4 to 8 nucleotides long. The use of a non-complementary portion is not preferred when random or partially random primers are used for example, in whole genome amplification.

The non-complementary portion of a primer can include sequences to be used to further manipulate or analyze amplified sequences. An example of such a sequence is a detection tag, which is a specific nucleotide sequence present in the non-complementary portion of a primer. Detection tags have sequences complementary to detection probes. Detection tags can be detected using their cognate detection probes. Detection tags become incorporated at the ends of amplified strands. The result is amplified DNA having detection tag sequences that are complementary to the complementary portion of detection probes. If present, there may be one, two, three, or more than three detection tags on a primer. It is preferred that a primer have one, two, three or four detection tags. Most preferably, a primer will have one detection tag. Generally, it is preferred that a primer have 10 detection tags or less. There is no fundamental limit to the number of detection tags that can be present on a primer except the size of the primer. When there are multiple detection tags, they may have the same sequence or they may have different sequences, with each different sequence complementary to a different detection probe. It is preferred that a primer comprise detection tags that have the same sequence such that they are all complementary to a single detection probe. For some multiplex detection methods, it is preferable that primers comprise up to six detection tags and that the detection tag portions have different sequences such that each of the detection tag portions is complementary to a different detection probe. A similar effect can be achieved by using a set of primers where each has a single different detection tag. The detection tags can each be any length that supports specific and stable hybridization between the detection tags and the detection probe. For this purpose, a length of about 10 to about 35 nucleotides is preferred, with a detection tag portion about 15 to about 20 nucleotides long being most preferred.

Address Tag

Another example of a sequence that can be included in the non-complementary portion of a primer is an address tag. An address tag has a sequence complementary to an address probe. Address tags become incorporated at the ends of amplified strands. The result is amplified DNA having address tag sequences that are complementary to the complementary portion of address probes. If present, there may be one, or more than one, address tag on a primer. It is preferred that a primer have one or two address tags. Most preferably, a primer will have one address tag. Generally, it is preferred that a primer have 10 address tags or less. There is no fundamental limit to the number of address tags that can be present on a primer except the size of the primer. When there are multiple address tags, they may have the same sequence or they may have different sequences, with each different sequence complementary to a different address probe. It is preferred that a primer comprise address tags that have the same sequence such that they are all complementary to a single address probe. The address tag portion can be any length that supports specific and stable hybridization between the address tag and the address probe. For this purpose, a length between about 10 and 35 nucleotides long is preferred, with an address tag portion between about 15 to 20 nucleotides long being most preferred.

Detection Labels

The oligomer can comprise a photochemically active group, a thermochemically active group, a chelating group, a reporter group, or a ligand that facilitates the direct of indirect detection of the oligomer or the immobilization of the oligomer onto a solid support. Such groups are typically attached to the oligonucleotide when it is intended as a probe for in situ hybridization, in Southern hybridization, Dot blot hybridization, reverse Dot blot hybridization, or in Northern hybridization.

When the photochemically active group, the thermochemically active group, the chelating group, the reporter group, or the ligand includes a spacer, the spacer may suitably comprise a chemically cleavable group.

In the present context, the term “photochemically active groups” covers compounds which are able to undergo chemical reactions upon irradiation with light. Illustrative examples of functional groups hereof are quinones, especially 6-methyl-1,4-naphtoquinone, anthraquinone, naphtoquinone, and 1,4-dimethyl-anthraquinone, diazirines, aromatic azides, benzophenones, psoralens, diazo compounds, and diazirino compounds.

In the present context “thermochemically reactive group” is defined as a functional group which is able to undergo thermochemically-induced covalent bond formation with other groups. Illustrative examples of functional parts thermochemically reactive groups are carboxylic acids, carboxylic acid esters such as activated esters, carboxylic acid halides such as acid fluorides, acid chlorides, acid bromide, and acid iodides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, and boronic acid derivatives.

In the present context, the term “chelating group” means a molecule that contains more than one binding site and frequently binds to another molecule, atom or ion through more than one binding site at the same time. Examples of functional parts of chelating groups are iminodiacetic acid, nitrilotriacetic acid, ethylenediamine tetraacetic acid (EDTA), aminophosphonic acid, etc.

In the present context, the term “reporter group” means a group which is detectable either by itself or as a part of an detection series. Examples of functional parts of reporter groups are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are dansyl (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO(N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radioisotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy), enzymes (such as peroxidases, alkaline phosphatases, α-galactosidases, and glycose oxidases), antigens, antibodies, haptens (groups which are able to combine with an antibody, but which cannot initiate an immune response by itself, such as peptides and steroid hormones), carrier systems for cell membrane penetration such as: fatty acid residues, steroid moieties (cholesteryl), vitamin A, vitamin D, vitamin E, folic acid peptides for specific receptors, groups for mediating endocytose, epidermal growth factor (EGF), bradykinin, and platelet derived growth factor (PDGF). Especially interesting examples are biotin, fluorescein, Texas Red, rhodamine, dinitrophenyl, digoxigenin, Ruthenium, Europium, Cy5, Cy3, etc.

In the present context “ligand” is a molecule, such as an antibody, hormone, or drug, that binds to a receptor. A ligand can comprise a molecule, ion, or atom that is bonded to the central metal atom of a coordination compound. Ligands can comprise functional groups such as: aromatic groups (such as benzene, pyridine, naphthalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulfur atoms, optionally containing aromatic or mono/polyunsaturated hydrocarbons, polyoxyethylene such as polyethylene glycol, oligo/polyamides such as poly-α-alanine, polyglycine, polylysine, peptides, oligo/polysaccharides, oligo/polyphosphates, toxins, antibiotics, cell poisons, and steroids, and also “affinity ligands”, i.e. functional groups or biomolecules that have a specific affinity for sites on particular proteins, antibodies, poly- and oligosaccharides, and other biomolecules.

It should be understood that the above-mentioned specific examples under DNA intercalators, photochemically active groups, thermochemically active groups, chelating groups, reporter groups, and ligands correspond to the “active/functional” part of the groups in question. For the person skilled in the art it is furthermore clear that DNA intercalators, photochemically active groups, thermochemically active groups, chelating groups, reporter groups, and ligands are typically represented in the form M-K- where M is the “active/functional” part of the group in question and where K is a spacer through which the “active/functional” part is attached to the 5- or 6-membered ring. Thus, it should be understood that the group B, in the case where B is selected from DNA intercalators, photochemically active groups, thermochemically active groups, chelating groups, reporter groups, and ligands, has the form M-K-, where M is the “active/functional” part of the DNA intercalator, photochemically active group, thermochemically active group, chelating group, reporter group, and ligand, respectively, and where K is an optional spacer comprising 1-50 atoms, preferably 1-30 atoms, in particular 1-15 atoms, between the 5- or 6-membered ring and the “active/functional” part.

In the present context, the term “spacer” means a thermochemically and photochemically non-active distance-making group and is used to join two or more different moieties of the types defined above. Spacers are selected on the basis of a variety of characteristics including their hydrophobicity, hydrophilicity, molecular flexibility and length (e.g. see Hermanson et. al., “Immobilized Affinity Ligand Techniques”, Academic Press, San Diego, Calif. (1992), p. 137-ff). Generally, the length of the spacers are less than or about 400 Å, in some applications preferably less than 100 Å. The spacer, thus, comprises a chain of carbon atoms optionally interrupted or terminated with one or more heteroatoms, such as oxygen atoms, nitrogen atoms, and/or sulfur atoms. Thus, the spacer K may comprise one or more amide, ester, amino, ether, and/or thioether functionalities, and optionally aromatic or mono/polyunsaturated hydrocarbons, polyoxyethylene such as polyethylene glycol, oligo/polyamides such as poly-α-alanine, polyglycine, polylysine, and peptides in general, oligosaccharides, oligo/polyphosphates. Moreover the spacer may consist of combined units thereof. The length of the spacer may vary, taking into consideration the desired or necessary positioning and spatial orientation of the “active/functional” part of the group in question in relation to the 5- or 6-membered ring. In particularly interesting embodiments, the spacer includes a chemically cleavable group. Examples of such chemically cleavable groups include disulphide groups cleavable under reductive conditions, peptide fragments cleavable by peptidases, etc.

Oligonucleotides of the invention may be used in high specificity oligo arrays, including, but not limited to, for example, wherein a multitude of different oligonucleotides are affixed to a solid surface in a predetermined pattern (Nature Genetics, suppl. vol. 21, January 1999, 1-60 and WO 96/31557); amplification libraries can be used for array screening and the information derived from such a library may be utilized during the array production. The usefulness of such an array, which can be used to simultaneously analyze a large number of target nucleic acids, depends to a large extend on the specificity of the individual oligonucleotides bound to the surface. The target nucleic acids may carry a detectable label or be detected by incubation with suitable detection probes which may also be an oligonucleotide of the invention.

An additional object of the present invention is to provide oligonucleotides which combines an increased ability to discriminate between complementary and mismatched targets with the ability to act as substrates for nucleic acid active enzymes such as for example DNA and RNA polymerases, ligases, phosphatases. Such oligonucleotides may be used for instance as primers for sequencing nucleic acids and as primers in the amplification reactions described herein.

In a further aspect, oligonucleotides of the invention may be used to construct new affinity pairs with exhibit enhanced specificity towards each other. The affinity constants can easily be adjusted over a wide range and a vast number of affinity pairs can be designed and synthesized. One part of the affinity pair can be attached to the molecule of interest (e.g. proteins, amplicons, enzymes, polysaccharides, antibodies, haptens, peptides, etc.) by standard methods, while the other part of the affinity pair can be attached to e.g. a solid support such as beads, membranes, micro-titer plates, sticks, tubes, etc. The solid support may be chosen from a wide range of polymer materials such as for instance polypropylene, polystyrene, polycarbonate or polyethylene. The affinity pairs may be used in selective isolation, purification, capture and detection of a diversity of the target molecules.

To further aid in detection and quantitation of nucleic acids amplified using the disclosed method, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY™, Cascade Blue™, Oregon Green™, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodarmine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodanine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Preferred fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a preferred form of detection label since they can be directly incorporated into the amplification products during synthesis. Examples of detection labels that can be incorporated into amplified nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other preferred nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A preferred nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.1.1.sup.3,7]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

The methods and/or compositions disclosed herein, can be used to generate amplification libraries for array screening and the information derived from such a library can be utilized during the array production. For example, oligonucleotide libraries may be employed as probes in the purification, isolation and detection of for instance pathogenic organisms such as viral, bacteria and fungi etc. Oligonucleotides also may be used as generic tools for the purification, isolation, amplification and detection of nucleic acids from groups of related species such as for instance rRNA from gram-positive or gram negative bacteria, fungi, mammalian cells etc.

The methods and/or compositions disclosed herein, can be used to generate aptamers in molecular diagnostics, e.g. in RNA mediated catalytic processes, in specific binding of antibiotics, drugs, amino acids, peptides, structural proteins, protein receptors, protein enzymes, saccharides, polysaccharides, biological cofactors, nucleic acids, or triphosphates or in the separation of enantiomers from racemic mixtures by stereospecific binding.

The methods and/or compositions disclosed herein, can be used for labeling of cells, e.g. in methods wherein the label allows the cells to be separated from unlabelled cells.

The methods and/or compositions disclosed herein, can be used to generate amplification libraries and the amplified nucleic acid molecules can be conjugated to a compound selected from proteins, amplicons, enzymes, polysaccharides, antibodies, haptens, and peptides. Any method well known to one of ordinary skill in the art can be used.

Nucleic Acid Fingerprints

The disclosed method can be used to produce replicated strands that serve as a nucleic acid fingerprint of a complex sample of nucleic acid. Such a nucleic acid fingerprint can be compared with other, similarly prepared nucleic acid fingerprints of other nucleic acid samples to allow convenient detection of differences between the samples. The nucleic acid fingerprints can be used both for detection of related nucleic acid samples and comparison of nucleic acid samples. For example, the presence or identity of specific organisms can be detected by producing a nucleic acid fingerprint of the test organism and comparing the resulting nucleic acid fingerprint with reference nucleic acid fingerprints prepared from known organisms. Changes and differences in gene expression patterns can also be detected by preparing nucleic acid fingerprints of mRNA from different cell samples and comparing the nucleic acid fingerprints. The replicated strands can also be used to produce a set of probes or primers that is specific for the source of a nucleic acid sample. The replicated strands can also be used as a library of nucleic acid sequences present in a sample. Nucleic acid fingerprints can be made up of, or derived from, for example, whole genome amplification of a sample such that the entire relevant nucleic acid content of the sample is substantially represented, or from multiple strand displacement amplification of selected target sequences within a sample.

Nucleic acid fingerprints can be stored or archived for later use. For example, replicated strands produced in the disclosed method can be physically stored, either in solution, frozen, or attached or adhered to a solid-state substrate such as an array. Storage in an array is useful for providing an archived probe set derived from the nucleic acids in any sample of interest. As another example, informational content of, or derived from, nucleic acid fingerprints can also be stored. Such information can be stored, for example, in or as computer readable media. Examples of informational content of nucleic acid fingerprints include nucleic acid sequence information (complete or partial); differential nucleic acid sequence information such as sequences present in one sample but not another; hybridization patterns of replicated strands to, for example, nucleic acid arrays, sets, chips, or other replicated strands. Numerous other data that is or can be derived from nucleic acid fingerprints and replicated strands produced in the disclosed method can also be collected, used, saved, stored, and/or archived.

Nucleic acid fingerprints can also comprise or be made up of other information derived from the information generated in the disclosed method, and can be combined with information obtained or generated from any other source. The informational nature of nucleic acid fingerprints produced using the disclosed method lends itself to combination and/or analysis using known bioinformatics systems and methods.

Nucleic acid fingerprints of nucleic acid samples can be compared to a similar nucleic acid fingerprint derived from any other sample to detect similarities and differences in the samples (which is indicative of similarities and differences in the nucleic acids in the samples). For example, a nucleic acid fingerprint of a first nucleic acid sample can be compared to a nucleic acid fingerprint of a sample from the same type of organism as the first nucleic acid sample, a sample from the same type of tissue as the first nucleic acid sample, a sample from the same organism as the first nucleic acid sample, a sample obtained from the same source but at time different from that of the first nucleic acid sample, a sample from an organism different from that of the first nucleic acid sample, a sample from a type of tissue different from that of the first nucleic acid sample, a sample from a strain of organism different from that of the first nucleic acid sample, a sample from a species of organism different from that of the first nucleic acid sample, or a sample from a type of organism different from that of the first nucleic acid sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same DNA molecule, or the same DNA library. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal, different mono-cellular or multi-cellular organisms. Different organism includes a different organism of the same type or organisms of different types. A different type of organism refers to organisms of different types such as a dog and cat, a human and a mouse, or bacteria such as E. coli and Salmonella. A different type of tissue refers to tissues of different types such as liver and kidney, or skin and brain. A different strain or species of organism refers to organisms differing in their species or strain designation as those terms are understood in the art.

Solid-State Detectors

Solid-state detectors are solid-state substrates or supports to which address probes or detection molecules have been coupled. A preferred form of solid-state detector is an array detector. An array detector is a solid-state detector to which multiple different address probes or detection molecules have been coupled in an array, grid, or other organized pattern.

Solid-state substrates for use in solid-state detectors can include any solid material to which oligonucleotides can be coupled. This includes materials such as acrylamide, cellulose, nitrocellulose, glass, gold, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin films or membranes, beads, bottles, dishes, fibers, optical fibers, woven fibers, chips, compact disks, shaped polymers, particles and microparticles. A chip is a rectangular or square small piece of material. Preferred forms for solid-state substrates are thin films, beads, or chips.

Address probes immobilized on a solid-state substrate allow capture of the products of the disclosed amplification method on a solid-state detector. Such capture provides a convenient means of washing away reaction components that might interfere with subsequent detection steps. By attaching different address probes to different regions of a solid-state detector, different amplification products can be captured at different, and therefore diagnostic, locations on the solid-state detector. For example, in a multiplex assay, address probes specific for numerous different amplified nucleic acids (each representing a different target sequence amplified via a different set of primers) can be immobilized in an array, each in a different location. Capture and detection will occur only at those array locations corresponding to amplified nucleic acids for which the corresponding target sequences were present in a sample.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol. Biol. (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994). Examples of nucleic acid chips and arrays, including methods of making and using such chips and arrays, are described in U.S. Pat. Nos. 6,287,768, 6,288,220, 6,287,776, 6,297,006, and 6,291,193 which are hereby incorporated by reference in their entirety.

Address Probes

An address probe is an oligonucleotide having a sequence complementary to address tags on primers. The complementary portion of an address probe can be any length that supports specific and stable hybridization between the address probe and the address tag. For this purpose, a length of about 10 to 35 nucleotides is preferred, with a complementary portion of an address probe about 12 to 18 nucleotides long being most preferred. An address probe can contain a single complementary portion or multiple complementary portions. Preferably, address probes are coupled, either directly or via a spacer molecule, to a solid-state support. Such a combination of address probe and solid-state support are a preferred form of solid-state detector.

Oligonucleotide Synthesis

Primers, detection probes, address probes, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et al. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. Nos. 6,294,664 and 6,291,669.

The nucleotide sequence of an oligonucleotide is generally determined by the sequential order in which subunits of subunit blocks are added to the oligonucleotide chain during synthesis. Each round of addition can involve a different, specific nucleotide precursor or a mixture of one or more different nucleotide precursors. In general, degenerate or random positions in an oligonucleotide can be produced by using a mixture of nucleotide precursors representing the range of nucleotides that can be present at that position. Thus, precursors for A and T can be included in the reaction for a particular position in an oligonucleotide if that position is to be degenerate for A and T. Precursors for all four nucleotides can be included for a fully degenerate or random position. Completely random oligonucleotides an be made by including all four nucleotide precursors in every round of synthesis. Degenerate oligonucleotides can also be made having different proportions of different nucleotides. Such oligonucleotides can be made, for example, by using different nucleotide precursors, in the desired proportions, in the reaction.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

Kits.

Kits are also provided containing one or more oligonucleotides of the invention for the isolation, purification, amplification, detection, identification, quantification, or capture of natural or synthetic nucleic acids. The kit typically will contain a reaction body, e.g. a slide or biochip. One or more oligonucleotides of the invention may be suitably immobilized on such a reaction body.

In another preferred embodiment, the invention provides a composition for amplifying RNA molecules comprising an isolated nucleic acid molecule from a sample; a modified oligo-dT primer and SWITCH primer comprising an RNA polymerase site; polymerase for amplification in a polymerase chain reaction.

In another preferred embodiment, a composition comprises a modified oligo-dT primer and SWITCH primer comprising an RNA polymerase site; a polymerase for amplification in a first-strand synthesis reaction, wherein, said polymerase adds nucleotides to 3′ ends of transcribed cDNA providing complementary nucleotides for the SWITCH primer.

In another preferred embodiment, the invention provides a kit, said kit comprising an isolated nucleic acid molecule from a sample; a modified oligo-dT primer and SWITCH primer comprising an RNA polymerase site; polymerase for amplification in a polymerase chain reaction. Preferably, instructions for use are included. Said instructions include a method, for example: amplification of nucleic acid molecules from a sample comprises the steps of: isolating nucleic acid molecules from a sample; providing a modified oligo-dT primer and SWITCH primer comprising an RNA polymerase site; hybridizing said isolated nucleic acid sample and said primers; administering polymerase for amplification in a first-strand synthesis reaction, wherein, said polymerase adds nucleotides to 3′ ends of transcribed cDNA providing complementary nucleotides for the SWITCH primer; and, creating an extended template for said SWITCH primer wherein said polymerase switches templates and amplifies said template; thereby, providing full-length, single stranded cDNA comprising a complete 5′ end of isolated nucleic acid, as well as sequences that are complimentary to the T7-SWITCH oligonucleotide; and, cycling of steps thereby, amplifying said genes.

The invention also provides methods for using kits of the invention for carrying out a variety of bioassays. Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

### EXAMPLES

Materials and Methods

Total RNA was isolated from LN 229 cells, primary glioma cells, and embryonic stem/progenitor cells (Clonetics) utilizing RNeasy Mini Kit (Qiagen) with DNAse I treatment on the column. The quality of extracted total RNA was confirmed using HP 2100 Bioanalyzer (Agilent Technologies). Different amounts of total RNA were used for the amplification procedure: 0.5 ng, 1 ng, 2 ng, 5 ng, 20 ng. A modified oligo-dT primer and T7-SWITCH primer bearing T7 RNA polymerase site were used in the first-strand synthesis reaction.

For each reaction 0.5 μl of 10 μM modified oligo-dT primer and 0.5 μl of 10 μM T7-SWITCH oligonucleotide was added to total RNA with 5 μl of final volume. The tubes were incubated at 72° C. for 2 min and then chilled on ice. The first strand buffer (Invitrogen) (25 mM Tris-HCl, pH 8.3; 37.5 mM KCl; 1.5 mM MgCl₂), 1 mM dNTP (CLONTECH), 2 mM DTT (Invitrogen), 3 mM MgCl₂ (Sigma), was added to each tube (all concentrations are final). The tubes were incubated at 42° C. for 5 min, then 1 μl of Superscript (Invitrogen) was added and tubes were incubated at 42° C. additionally for 1 hour. The final volume of reaction was 10 μl.

Purification

Two purification approaches were utilized for 1^{st} strand cDNA cleaning. The first method is using phenol/chloroform/isoamyl alcohol (PCI). RNAse-free water (Ambion) was added to 1^{st} strand synthesis reaction to bring final volume to 200 μl. For extraction of the cDNA pool, 200 μl of PCI (25:24:1) was added followed by centrifugation at 7,000 g for 5 min at room temperature. The aqueous phase was transferred to a new tube. The PCI procedure was repeated twice. 1/10 volume of 5 M ammonium acetate and 2.5 volume of 96% Ethanol, and 20 μg of GenElute (Sigma), LPA (Linear Polyacrylamide) were added as a carrier to combined aqueous phases for overnight precipitation at −20° C. The another approach utilized Microcon, YM-30 columns (Millipore). The manufacturer's instruction were followed during this purification procedure.

The purified and concentrated first-strand cDNA pool produced at the previous step was then subjected to LD (Long Distance) amplification using the Advantage 2 PCR Enzyme System (CLONTECH) and Amplification primer to produce double-stranded cDNA as recommended by the manufacturer. An MJR PCR machine was used for amplification with the following parameters: 95° C. for 1 min; (95° C. for 15 sec, 65° C. for 30 sec, 68° C. for 6 min)—12 cycles. To remove RNA before in vitro transcription 7.5 μl of 1 M NaOH, 2 mM EDTA was added to each tube after double-strand synthesis step. The tube was incubated at 65° C. for 5 min. The alkaline hydrolysis of RNA was followed by neutralization with 0.5 M Tris-HCl (pH 6.8). The double-stranded DNA pool bearing T7 Promoter was purified using QIAqiuck PCR Purification Kit (Qiagen) and concentrated on Microcon, YM-30 columns (Millipore) as recommended by the manufacturers.

Sense RNA was transcribed from double-stranded DNA using reagents from MEGAScript High Yield Transcription Kit (Ambion). 12 μl of 75 mM NTP, 3 μl of 10× Buffer, and 3 μl of Enzyme Mix (RNase Inhibitor and T7 RNA Polymerase) were added to 12 μl of double-stranded DNA. The mixture was incubated for 18 h at 37° C. Upon completion, the reaction was treated with 2 μl of DNase I (Ambion) for 30 min at 37° C. The amplified sense RNA was purified with MEGAclear Kit (Ambion) as described in the Ambion instruction manual. The RNA yield was evaluated by SmartSpec 3000 (BioRad). This method of RNA amplification produces 20-50 μg amplified sense RNA after one round which is enough for any downstream application.

### Example 1

### Amplification of Oligonucleotides

There is the limited amount of total RNA presented in a single cell (20-30 pg). The two current methods do not provide unlimited amplification since PCR has a natural limitation (the so-called “plateau effect”), and the aRNA method results in a 10⁵-10⁶-fold amplification of 3′ biased RNA after two rounds. Micrograms of RNA are needed for cDNA array screening, subtraction procedures, quantitative PCR, reverse Northerns, etc. The present method affords an almost unlimited linear RNA amplification from a few cells with minimal differences in the relative abundance of amplified RNAs and their parent mRNA (sample distortion). This RNA amplification procedure creates a regenerating biorepository that represents the complex mRNA profile of the original sample (FIG. 1—flow chart). The procedure exploits the template switching activity of reverse transcriptase (RT) to incorporate RNA polymerase binding sites upstream of single stranded DNA (ssDNA). Limited amounts of PCR cycles, as low as 12-13 cycles are used for the synthesis of double-stranded (dsDNA) in order to introduce minimal sample distortion. The resultant template is a subject of T7 RNA Polymerase amplification, yielding 1.7×10⁴-8.3×10⁴ fold amplification after one round and 5.8×10⁶-2.4×10⁷ fold amplification after two rounds. The calculations were made based on UV readings.

To characterize the amplified product, a novel approach was utilized via taking into the consideration the different length, structure and the abundance of specific transcripts. The candidate genes chosen represent all groups of abundance designated herein as: 1) abundant (approximately 1,000-3,000 copies per cell); 2) moderate expressed genes (300-1,000 copies per cell); 3) rare genes (less than 300 copies per cell). Each group should contain the transcripts which differ in the length (small, medium, long). The human glioma LN 229 cell line has been chosen as a test sample. Total RNA was isolated from cells growing on six plates (50-70% of confluent), which provides enough material to compare the amplified and the original sample. The LN 229 population was propagated from a single cell. The quality of isolated total RNA was confirmed using an Agilent 2100 Bioanalyzer (FIG. 2-4, samples 051, 052-total). Four transcripts were selected to characterize the samples with or without a round(s) of amplification using real-time PCR. Two of them belong to the group of abundant genes: actin (the length is 1,800 nt; 3,200 copies per cell) and tenascin (the length is 7,500 nt; 1,100 copies per cell). Other candidates are rare genes: TBP (the length is 1,900 nt; 76 copies per cell) and TFRC (the length is 5,000 nt, 160 copies per cell). The approximate copy number was calculated based on the calibration curve of each given transcript. To achieve this, full-length transcripts were amplified by PCR using proof-reading DNA Polymerase. This approach allows monitoring 5′/3′ ratio of amplified products. To further describe this amplification system, more markers (up to nine) from the aforementioned groups are applied.

The different amounts of total RNA from the LN 229 cell line, as well from single glioma-derived “neurospheres”, were taken for the amplification procedure: 0.5 ng, 1 ng, 2 ng, 5 ng, 20 ng. A modified oligo-dT primer and T7-SWITCH primer bearing T7 RNA polymerase site were used in the first-strand synthesis reaction (FIG. 1—Flow Chart). When reverse transcriptase reaches the 5′ end of mRNA, the enzyme's terminal transferase activity adds a few additional nucleotides, primarily deoxycitidine, to the 3′ end of the cDNA. The T7-SWITCH primer, which has an oligo (rG) sequence at its 3′ end, base-pairs with the deoxycitidine stretch, creating an extended template. RT then switches templates and continues replicating to the end of the oligonucleotide. The resulting full-length, single stranded cDNA contains the complete 5′ end of the mRNA, as well as sequences that are complimentary to the T7-SWITCH oligonucleotide. The T7-SWITCH anchor sequence and the polyA sequence serve as universal priming sites for end-to-end cDNA amplification via long-distance PCR using only one specific primer. The dsDNA contains a T7 RNA Polymerase site which is used for the production of sense RNA during T7 Polymerase amplification step. The resultant sense RNA can be a subject of the second round of the amplification (FIG. 1—Flow Chart). FIG. 1 is a schematic illustration of the method described herein.

The expression profile of different amounts of total RNA from LN 229 (0.5-20 ng range) that are amplified resembles that of the sample without amplification (FIGS. 5-10). Total RNA (1,860 ng) of the original sample was taken for RT without amplification round(s). To mimic the isolation of RNA from small numbers of cells, 1 ng of total RNA from LN 229 was taken through the RNA isolation process again. The isolation of nucleic acids from a few cells led to a dramatic loss of material (up to 50-75%). The approximately 250 pg of total RNA after isolation was split into two tubes for further amplification. This amount of total RNA corresponds to 4-6 cells—if one cell contains 20-30 pg of total RNA, 5 cells contain approximately 125 pg.

A comparison of one and two rounds of amplification yields similar amplification results (FIGS. 9 and 11, 12 and 14, 13 and 15) that can be explained by the similarity of amplification approaches and the template's identity (sense RNA) used in the first and the second rounds. In contrast, the aRNA amplification methods, classic and modified, utilize different primers in every round and different types of template: sense—for the first round, antisense—for the second.

One of the major advantages of the method disclosed herein, is that short transcripts from a rare gene group do not become abundant genes after amplification, and do not overexpress the long abundant transcripts, and vice versa. Optimum numbers of cycles were determined. A large distortion was shown when the amplification products from single glioma spheres were obtained using 21 cycles of PCR as compared to 12 cycles (FIGS. 12 and 16, 13 and 17).

The present method utilizes: 5′ end enrichment, and limited amplification by a SMART-like method that prevents template loss when compared to T7 RNA amplification, providing a high template-yield. The linearity of amplification by DNA-dependent RNA Polymerase and its processivity provide the conservation of a minimal sample's distortion introduced by PCR. Methods known in the art require higher amounts of genetic material and produce lower yields. A method has recently been published (Rajeevan, et al., Genomics, 2003) where the investigators used 900-1000 ng of total RNA, yielding 2×10³-2.5×10³ fold amplification in comparison with 0.5 ng (up to 10⁵ fold) for the present system. Furthermore, 18 cycles of PCR utilized by other investigators during the long-distance step could introduce additional sample distortion, especially for rare transcripts. Moreover two different primers were used during PCR that may contribute to non-specific amplification and decrease the efficiency of long-distance PCR. In contrary, the system described here utilizes only one amplification primer.

All references cited herein, are incorporated by reference.

What is claimed is:

1. A method for amplifying varying copy numbers of genes comprising the steps of:
(a) isolating nucleic acid molecules from a sample;
(b) providing a modified oligo-dT primer and SWITCH primer comprising an RNA polymerase site;
(c) hybridizing said isolated nucleic acid sample and said primers;
(d) administering polymerase for amplification in a first-strand synthesis reaction, wherein, said polymerase adds nucleotides to 3′ ends of transcribed cDNA providing complementary nucleotides for the SWITCH primer; and,
(e) creating an extended template for said SWITCH primer wherein said polymerase switches templates and amplifies said template; thereby,
(f) providing full-length, single stranded cDNA comprising a complete 5′ end of isolated nucleic acid, as well as sequences that are complimentary to the T7-SWITCH oligonucleotide; and,
(g) cycling of steps (e) through (f) thereby, amplifying said genes.

2. The method of claim 1, wherein the isolated nucleic acid is RNA.

3. The method of claim 1, wherein the polymerase is RNA polymerase.

4. The method of claim 3, wherein the RNA polymerase is a T7 RNA polymerase.

5. The method of claim 1, wherein the SWITCH primer is a T7-SWITCH primer comprising a T7 RNA polymerase site and/or anchor site.

6. The method of claim 1, wherein the polymerase inserts at least one additional nucleotide at 3′ ends of transcribed mRNA.

7. The method of claim 1, wherein the polymerase inserts about two additional nucleotides at 3′ ends of transcribed mRNA.

8. The method of claim 1, wherein the polymerase inserts about 5 additional nucleotides at 3′ ends of transcribed mRNA.

9. The method of claim 1, wherein the polymerase inserts about 10 additional nucleotides at 3′ ends of transcribed mRNA.

10. The method of claim 1, wherein the polymerase inserts from about 2 up to 20 additional nucleotides at 3′ ends of transcribed mRNA.

11. The method of claim 10, wherein the inserted nucleotides at the 3′ ends of transcribed mRNA are deoxycitidine.

12. The method of claim 1, wherein the SWITCH primer comprises an oligonucleotide sequence at said primer's 3′ end.

13. The method of claim 1, wherein the SWITCH primer comprises an oligonucleotide sequence at said primer's 3′ end which are complementary to the inserted nucleotides at the 3′ ends of transcribed mRNA.

14. The method of claim 13, wherein the SWITCH primer comprises a guanosine oligonucleotide sequence at said primer's 3′ end.

15. The method of claim 13, wherein the guanosine oligonucleotide sequence base-pairs with the deoxycitidine nucleotides at the 3′ ends of transcribed mRNA.

16. The method of claim 5, wherein the T7-SWITCH anchor sequence and poly A sequence are universal priming sites for end-to-end cDNA amplification via long-distance PCR.

17. The method of claim 1, wherein a sequence specific primer and/or universal base primer are administered to the amplification method at step (b).

18. The method of claim 1, wherein amplification cycles of isolated nucleic acid sequences are between about 10 to 20 cycles.

19. The method of claim 1, wherein one cycle of amplification yields between about 1.7×10⁴-8.3×10⁴ fold of amplified product as compared to controls which have not been subjected to amplification.

20. The method of claim 1, wherein two cycles of amplification yields between about 5.8×10⁶-2.4×10⁷ fold of amplified product as compared to controls which have not been subjected to amplification.

21. The method of claim 19, wherein amplification yields are determined by U.V. readings.

22. The method of claim 1, wherein low copy numbers of genes are amplified.

23. The method of claim 1, wherein medium copy numbers of genes are amplified.

24. The method of claim 1, wherein high copy numbers of genes are amplified.

25. The method of claim 1, wherein the nucleic acid molecules are amplified with high fidelity with an error rate between about 10⁻⁶-10⁻¹⁰.

26. A method for identifying one or more rare genes in a mammal comprising:
amplifying nucleic acid molecules from a mammal by (a) isolating nucleic acid molecules from a mammal;
(b) providing a modified oligo-dT primer and SWITCH primer comprising an RNA polymerase site;
(c) hybridizing said isolated nucleic acid from a mammal and said primers;
(d) administering polymerase for amplification in a first-strand synthesis reaction, wherein, said polymerase adds nucleotides to 3′ ends of transcribed cDNA providing complementary nucleotides for the SWITCH primer; and,
(e) creating an extended template for said SWITCH primer wherein said polymerase switches templates and amplifies said template; thereby,
(f) providing full-length, single stranded cDNA comprising a complete 5′ end of isolated nucleic acid, as well as sequences that are complimentary to the T7-SWITCH oligonucleotide; and,
(g) cycling of steps (e) through (f) thereby, amplifying said genes;
hybridizing the isolated nucleic acid sequence with a nucleic acid probe to form a hybridized molecule; and,
detecting sequences hybridized to the probe.

27. The method of claim 26, wherein the amplified gene, allele or fragment oligopeptide are provided on a solid support.

28. The method of claim 26, wherein binding of the candidate gene and/or gene product with the amplified gene, allele or fragment or oligopeptide is detected.

29. The method of claim 26, wherein the amplified gene sequence is compared known genes in a database.

30. The method of claim 29, wherein the amplified gene is identified from the database.

31. The method of claim 30, wherein the database is GenBank, Human genome project or EMBL.

32. A composition for amplifying RNA molecules comprising:
an isolated nucleic acid molecule from a sample;
a modified oligo-dT primer and SWITCH primer comprising an RNA polymerase site;
polymerase for amplification in a polymerase chain reaction.

33. The composition of claim 32, wherein the modified oligo-dT primer comprises at least one modified nucleobase.

34. The composition of claim 32, wherein the modified oligo-dT primer comprises about two modified nucleobases.

35. The composition of claim 32, wherein the modified oligo-dT primer comprises about five modified nucleobases.

36. The composition of claim 32, wherein the modified oligo-dT primer comprises about ten modified nucleobases.

37. The composition of claim 32, wherein the modified oligo-dT primer comprises about 1, 2, 3, 4, 5, 6, 7, 8, 9, 10, or comprises only modified base units.

38. The composition of claim 32, wherein the modified oligo-dT primer comprises any one or combinations thereof, of phosphorthiorate, methylphosphonate, peptide nucleic acids, and LNA molecules.

39. The composition of claim 32 wherein the modified oligo-dT primer comprises between about two bases up to fifty nucleotide bases.

40. The composition of claim 32, wherein the SWITCH primer comprises a 3′ oligonucleotide stretch of guanosines (rG) of at least one nucleotide base up to twenty nucleotide bases.

41. The composition of claim 40, wherein the 3′ end of the SWITCH primer comprises about 1, 2, 3, 4, 5, 6, 7, 8, 9, 10 up to 15 guanosines (rG).

42. The composition of claim 40, wherein the 3′ end of the SWITCH primer comprises about 1, 2, 3, 4, 5, 6, 7, 8, 9, 10 modified guanosines (rG).

43. The composition of claim 32, wherein the SWITCH primer comprises at least five nucleotide bases up to fifty nucleotide bases.

44. A kit comprising:
a modified oligo-dT primer;
a SWITCH primer comprising an RNA polymerase site;
a polymerase for amplification in a polymerase chain reaction, and;
instructions for method of use.

45. The kit of claim 44, wherein the modified oligo-dT primer comprises at least one modified nucleobase.

46. The kit of claim 44, wherein the modified oligo-dT primer comprises about two modified nucleobases.

47. The kit of claim 44, wherein the modified oligo-dT primer comprises about five modified nucleobases.

48. The kit of claim 44, wherein the modified oligo-dT primer comprises about ten modified nucleobases.

49. The kit of claim 44, wherein the modified oligo-dT primer comprises about 1, 2, 3, 4, 5, 6, 7, 8, 9, 10, or comprises only modified base units.

50. The kit of claim 44, wherein the modified oligo-dT primer comprises any one or combinations thereof, of phosphorthiorate, methylphosphonate, peptide nucleic acids, and LNA molecules.

51. The kit of claim 44, wherein the modified oligo-dT primer comprises between about two bases up to fifty nucleotide bases.

52. The kit of claim 44, wherein the SWITCH primer comprises a 3′ oligonucleotide stretch of guanosines (rG) of at least one nucleotide base up to twenty nucleotide bases.

53. The kit of claim 44, wherein the 3′ end of the SWITCH primer comprises about 1, 2, 3, 4, 5, 6, 7, 8, 9, 10 up to 15 guanosines (rG).

54. The kit of claim 44, wherein the 3′ end of the SWITCH primer comprises about 1, 2, 3, 4, 5, 6, 7, 8, 9, 10 modified guanosines (rG).

55. The kit of claim 44, wherein the SWITCH primer comprises at least five nucleotide bases up to fifty nucleotide bases.
